(19) United States
(12) Patent Application Publication
(10) Pub. No.: US 2023/0279372 A1
(43) Pub. Date: Sep. 7, 2023

(54) METHODS TO CONTROL LIPOKINE CONCENTRATIONS AND USES THEREOF

(71) Applicant: Ohio State Innovation Foundation, Columbus, OH (US)

(72) Inventors: Kristin STANFORD, Dublin, OH (US); Daniel GALLEGO PEREZ, Columbus, OH (US); Mark ZIOLO, Dublin, OH (US)

(21) Appl. No.: 18/018,981

(22) PCT Filed: Jul. 30, 2021
(86) PCT No.: PCT/US2021/043973

§ 371 Date: Jan. 31, 2023

Related U.S. Application Data
(60) Provisional application No. 63/059,663, filed on Jul. 31, 2020.

(51) Int. Cl. C12N 9/14; A61K 38/46; A61P 9/00

(52) U.S. Cl. CPC C12N 9/14; A61K 38/46; C12Y 303/02003; A61P 9/00

(57) ABSTRACT

The present disclosure relates to compositions for regulating lipokines and methods of use thereof.

### CROSS-REFERENCE TO RELATED APPLICATIONS

This application claims the benefit of U.S. Provisional Application No. 63/059,663, filed Jul. 31, 2020, which is expressly incorporated herein by reference.

### STATEMENT REGARDING FEDERALLY SPONSORED RESEARCH

This invention was made with government support under Grant Nos. R01 HL138738 and R01 AG060542 awarded by the National Institutes of Health. The government has certain rights in the invention.

### FIELD

The present disclosure relates to compositions and methods for regulating lipokines and uses thereof.

### BACKGROUND

Brown adipose tissue (BAT) is an important tissue for thermogenesis, making it a potential target to decrease the risks of obesity, type 2 diabetes, and cardiovascular disease (CVD), and recent studies have also identified BAT as an endocrine organ. To date, there are no studies that identify a direct role for BAT to mediate cardiac function. Therefore, what is needed are compositions and methods for treating CVD, obesity, and type 2 diabetes through the regulation of BAT.

### SUMMARY

Disclosed herein are compositions and uses thereof for increasing a level of a lipokine (e.g., 12,13-diHOME) in a subject in need thereof comprising administering to the subject an effective amount of an epoxide hydrolase (Ephx) polypeptide or a polynucleotide encoding the Ephx polypeptide. The increased level of the lipokine in the subject can improve cardiac function, mitigating symptoms of cardiovascular diseases, type 2 diabetes, and obesity.

Accordingly, in some aspects, disclosed herein is a method for increasing a level of a lipokine in a subject in need thereof, comprising administering to the subject an effective amount of an epoxide hydrolase (Ephx) polypeptide or a polynucleotide encoding the Ephx polypeptide.

In some embodiments, the Ephx polypeptide comprises an Ephx1 polypeptide or an Ephx2 polypeptide. In some embodiments, the Ephx polypeptide comprises an Ephx1 polypeptide and an Ephx2 polypeptide.

In some embodiments, the polynucleotide encoding the Ephx polypeptide comprises a nucleic acid sequence at least about 80% identical to SEQ ID NO:1, SEQ ID NO:3, SEQ ID NO:5, or SEQ ID NO:7.

In some embodiments, the Ephx polypeptide comprises an amino acid sequence at least about 80% identical to SEQ ID NO:2, SEQ ID NO:4, SEQ ID NO:6, SEQ ID NO:8, SEQ ID NO:9, SEQ ID NO:10, SEQ ID NO: 11, or SEQ ID NO:12.

In some embodiments, the polynucleotide is contained in a vector. In some embodiments, the vector is a viral vector. In some embodiments, the viral vector is an adeno-associated virus (AAV) vector.

In some embodiments, the polynucleotide is an mRNA. In some embodiments, the mRNA is contained in a nanoparticle.

In some embodiments, the lipokine comprises 12,13-diHOME.

In some aspects, disclosed herein is a method of treating a cardiovascular disease, comprising administering to the subject a therapeutically effective amount of an epoxide hydrolase (Ephx) polypeptide or a polynucleotide that encodes the Ephx polypeptide.

In some aspects, disclosed herein is a method of treating an inflammatory disease, comprising administering to the subject a therapeutically effective amount of an epoxide hydrolase (Ephx) polypeptide or a polynucleotide that encodes the Ephx polypeptide. In some embodiments, the inflammatory disease is type 2 diabetes or nonalcoholic fatty liver disease.

### BRIEF DESCRIPTION OF THE DRAWINGS

The accompanying figures, which are incorporated in and constitute a part of this specification, illustrate several aspects described below.

FIGS. 1A-1F show exercise or increasing BAT mass by transplantation improves cardiac function and structure in mice. (FIG. 1A) Systolic function and (FIG. 1B) diastolic function measured by in vivo cardiac hemodynamics (n=4-6/group). Cardiac function and structure measured by (FIG. 1C) ejection fraction, (FIG. 1D) end diastolic volume, (FIG. 1E) left ventricular mass, and (FIG. 1F) diastolic diameter. Data are mean±S.E.M (n=6/group). Asterisks represent difference vs. Sham-Sedentary (*P<0.05). Repeated measures two-way ANOVA was used for FIG. 1A and FIG. 1B with Tukey's multiple comparisons tests; one-way ANOVA was used for FIG. 1C, FIG. 1D, FIG. 1E, and FIG. 1F with Tukey's multiple comparisons tests.

FIGS. 2A-2F show exercise or transplantation of BAT increases circulating 12,13-diHOME, 9,10-diHOME, and 9-HODE. (FIG. 2A) Heat map and (FIG. 2B, 2C) volcano plot representing 88 lipids comparing the fold induction of Sham-Sedentary to the p value; 12,13-diHOME is circled in red. Data are mean±S.E.M (n=6/group). Plasma concentrations of (FIG. 2D) 12,13-diHOME, (FIG. 2E) 9,10-diHOME, and (FIG. 2F) 9-HODE. Data are mean±S.E.M (n=6/group). Asterisks represent difference vs. Sham-Sedentary (*P<0.05; **P<0.01; ***P<0.001). One-way ANOVA was used for FIG. 2C, FIG. 2D, and FIG. 2E with Tukey's multiple comparisons tests.

FIGS. 3A-3N show that 12,13-diHOME improves in vivo cardiac function and structure. (FIG. 3A) Systolic and (FIG. 3B) diastolic function in mice acutely injected with saline (n=3), 12,13-diHOME (n=8), 9-HODE (n=5), or 9,10-diHOME (n=3). Data are mean+S.E.M Asterisks represent difference compared to mice injected with saline, 9-HODE, or 9,10-diHOME (*P<0.05; **P<0.01). (FIG. 3C) Systolic and (FIG. 3D) diastolic function measured by in vivo cardiac hemodynamics in Sham or +BAT mice fed the sEH inhibitor AUDA. (FIG. 3E) Systolic and (FIG. 3F) diastolic function measured by in vivo cardiac hemodynamics in Sham or +BAT mice fed the sEH inhibitor t-AUCB. Data are mean+S.E.M (n=6/group). (FIG. 3G) 12,13-diHOME in Baseline (n=8), Sham (n=5), TNT-Ephx1/2 (n=7), or TNT-Ucp1 (n=5) mice. Data are mean f S.E.M Asterisks represent difference compared to Sham (*P<0.05). Cardiac function and structure measured by (FIG. 3H) ejection fraction, (FIG. 3I) posterior wall thickness, (FIG. 3J) left ventricular mass, (FIG. 3K) diastolic diameter, and (FIG. 3L) end diastolic volume (EDV) was measured in Sham (n=5), TNT-Ephx1/2 (n=7), or TNT-Ucp1 (n=5). Data are mean±S.E.M Asterisks represent differences compared to baseline cohort (*P<0.05; **P<0.01). (FIG. 3M) Systolic function and (FIG. 3N) diastolic function measured by in vivo cardiac hemodynamics. Data are mean+S.E.M Asterisks represent difference in TNT-Ephx1/2 compared to all other groups (*P<0.05; **P<0.01; ***P<0.001). Repeated measures two-way ANOVA was used for FIGS. 3C-3F, FIG. 3M, and FIG. 3N with Tukey's multiple comparisons tests. One-way ANOVA was used for FIGS. 3G-3L with Tukey's multiple comparisons tests. Kruskal-Wallis test was used for FIG. 3A and FIG. 3B.

FIGS. 4A-4M show that 12,13-diHOME increases function and respiration in isolated cardiomyocytes. (FIG. 4A) Peak shortening, (FIG. 4B) Ca²⁺ transient, (FIG. 4C) maximal velocity of shortening, and (FIG. 4D) maximal velocity of relengthening in isolated cardiomyocytes±12,13-diHOME. Data are mean±S.E.M (n=4/group; 10-12 myocytes per mouse). Asterisks represent difference compared to vehicle (**P<0.01; ***P<0.001). (FIG. 4E) Fatty acid uptake in cardiomyocytes constitutively expressing firefly luciferase that were treated with either 12,13-diHOME or vehicle, as measured by luciferase activity using 10 μM FFA-SS-Luc. Data are mean f S.E.M (n=9 technical replicate wells per group. Asterisks represent difference compared to vehicle (**P<0.01). (FIG. 4F) Bioenergetic profile of cardiomyocytes treated with 12,13-diHOME or vehicle. (FIG. 4G) Basal OCR, (FIG. 4H) maximal respiration, and (FIG. 4I) non-mitochondrial respiration were measured. Data are mean±S.E.M (n=5/group). Asterisks represent differences compared to vehicle (*P<0.05; ***P<0.001). (FIG. 4J) Bioenergetic profile of cardiomyocytes treated with 12,13-diHOME or vehicle with or without BDM. (FIG. 4K) Basal OCR, (FIG. 4L) maximal respiration, and (FIG. 4M) non-mitochondrial respiration were measured. Data are mean±S.E.M (n=5/group). Asterisks represent differences compared to vehicle (*P<0.05; ***P<0.001), compared to vehicle+BDM (##P<0.01; ###P<0.001), or compared to 12,13-diHOME+BDM ($$$P<0.001). Unpaired two-tailed Student's t-test was used for FIGS. 4A-4D, 4G, 4H, and 4I. Two-way ANOVA was used for FIGS. 4E, 4F and 4J with Tukey's multiple comparisons tests; one-way ANOVA was used for FIGS. 4K, 4L, and 4M with Tukey's multiple comparisons tests.

FIGS. 5A-5O show that 12,13-diHOME improves cardiac function and respiration via NOS1 and RyR. (FIG. 5A) Systolic and (FIG. 5B) diastolic function in wild-type (WT) (n=3) or NOS1^{-/-} mice (n=4) acutely injected with 12,13-diHOME. Data are mean±S.E.M Asterisks represent difference compared WT (*P<0.05; **P<0.01). (FIG. 5C) Peak shortening, (FIG. 5D) Ca²⁺ transient, (FIG. 5E) maximal velocity of shortening, and (FIG. 5F) maximal velocity of relengthening in isolated cardiomyocytes from NOS1^{-/-} mice treated with PBS or 12,13-diHOME. Data are mean±S.E.M (n=2/group; 5-6 myocytes per mouse). (FIG. 5G) Bioenergetic profile of NOS1^{-/-} cardiomyocytes treated with 12,13-diHOME or vehicle. (FIG. 5H) Basal OCR, (FIG. 5I) maximal respiration, and (FIG. 5J) non-mitochondrial respiration were measured. Data are mean±S.E.M (n=5/group). (FIG. 5K) Bioenergetic profile of cardiomyocytes treated with 12,13-diHOME or vehicle with or without tetracaine. (FIG. 5L) Basal OCR, (FIG. 5M) maximal respiration, and (FIG. 5N) non-mitochondrial respiration were measured. Data are mean±S.E.M (n=5/group). Asterisks represent differences compared to vehicle (*P<0.05; ***P<0.001), compared to vehicle+tetracaine (#P<0.05; ###P<0.001), or compared to 12,13-diHOME+tetracaine ($$$P<0.001). (FIG. 5O) Proposed model for 12,13-diHOME to regulate cardiac function via NOS1. Unpaired two-tailed Student's t-test was used for FIGS. 5A-5J. Two-way ANOVA was used for FIG. 5G and FIG. 5K with Tukey's multiple comparisons tests. One-way ANOVA was used for FIG. 5L, FIG. 5M, and FIG. 5N with Tukey's multiple comparisons tests.

FIGS. 6A-6G show that 12,13-diHOME is decreased in human patients with heart disease. (FIG. 6A) Plasma concentrations of 12,13-diHOME of human subjects (healthy and heart disease). Data are mean±S.E.M (healthy males n=25; healthy female n=26; males with heart disease n=17; females with heart disease n=7). Asterisks represent differences compared to healthy controls of same gender (*P<0.05; **P<0.01), or an overall effect of heart disease (#P<0.05). (FIG. 6B) BMI among groups and (FIG. 6C) correlation among BMI and 12,13-diHOME. (FIG. 6D) Age among groups and (FIG. 6E) correlation among age and 12,13-diHOME. Asterisks represent differences among healthy male and female subjects (*P<0.05). In a subset of patients with heart disease, (FIG. 6F) ejection fraction and (FIG. 6G) fractional shortening correlated to 12,13-diHOME in plasma. One-way ANOVA was used for FIG. 6A, FIG. 6B, and FIG. 6D with Tukey's multiple comparisons tests. Spearman's correlation was used for FIG. 6C and FIG. 6E. Linear regression analyses were used for FIG. 6F and FIG. 6G.

FIGS. 7A-7L show exercise and +BAT have similar effects on metabolic health. (FIG. 7A) Contractility (dp/dt max normalized by EDV), (FIG. 7B) glucose tolerance test area under the curve (GTT AUC), (FIG. 7C) glucose excursion curve, (FIG. 7D) body weight, (FIG. 7E) % fat mass, (FIG. 7F) % lean mass, (FIG. 7G) V_{O2}, (FIG. 7H) V_{CO2}, (FIG. 7I) RER, (FIG. 7J) Heat/energy expenditure, and (FIG. 7K) activity in Sham-Sedentary, Sham-Exercised, or +BAT mice 12 wks after transplantation (8 wks of exercise). Data are mean±S.E.M (n=6/group). Asterisks represent difference vs. Sham-Sedentary (*P<0.05; **P<0.01; ***P<0.001). (FIG. 7L) Principal component analysis (PCA) of serum oxylipins from Shem-Sedentary, Sham-Exercised, or +BAT mice 12 wks after transplantation (8 wks of exercise). One-way ANOVA was used for FIG. 7B, FIG. 7D, FIG. 7E, FIG. 7F, FIG. 7G, FIG. 7H, FIG. 7I, and FIG. 7J with Tukey's multiple comparisons tests. Two-way ANOVA was used for FIG. 7A and FIG. 7C with Tukey's multiple comparisons tests.

FIGS. 8A-8L show effects of sustained treatment with 12,13-diHOME (TNT) metabolism and cardiac function. (FIG. 8A) Systolic and (FIG. 8B) diastolic function in mice acutely injected with saline (n=3), 12,13-diHOME (n=8), 9-HODE (n=5), or 9,10-diHOME (n=3). Data are mean±S.E.M. Asterisks represent difference compared to all other groups (*P<0.05; **P<0.01). (FIG. 8C) Gene expression data of Ucp1 in skin, BAT, and pgWAT of Sham and TNT-Ucp1 mice. Data are mean+S.E.M Asterisks represent difference compared to all other groups (*P<0.05). (FIG. 8D) Volcano plot representing 88 lipids comparing the fold induction of Sham and TNT-Ephx1/2 mice to the p value. (FIG. 8E) Systolic function and (FIG. 8F) diastolic function measured by in vivo cardiac hemodynamics. Data are mean±S.E.M (n=5-7/group). (FIG. 8G) Contractility (dp/dt max normalized per EDV) Data are mean±S.E.M (n=5-7/group). (FIG. 8H) Body weight, (FIG. 8I) % fat mass, and (FIG. 8J) % lean mass after 6 wks of TNT. (FIG. 8K) GTT AUC, and (FIG. 8L) glucose excursion curve after 6 wks of TNT. Asterisks represent difference compared to Sham (*P<0.05; **P<0.01; ***P<0.001); #represent differences compared to TNT-Ucp1 (#P<0.05). Paired two-tailed Student's t-test was used for FIG. 8A and FIG. 8B. Unpaired two-tailed Student's t-test was used for C. Two-way ANOVA was used for FIG. 8E, FIG. 8F, FIG. 8G, FIG. 8H, and FIG. 8K with Tukey's multiple comparisons tests. One-way ANOVA was used for FIG. 8I, FIG. 8J, and FIG. 8L with Tukey's multiple comparisons tests.

FIGS. 9A-9B show effects of acute treatment with 12,13-diHOME on cardiac function in NOS1^{-/-} mice. (FIG. 9A) Systolic and (FIG. 9B) diastolic function in WT mice (n=6) or NOS1^{-/-} mice (n=6) acutely injected with 12, 13-diHOME. Data are mean±S.E.M. Asterisks represent difference compared to pre-injection values (* P<0.05). Paired two-tailed Student's t-test was used for FIG. 9A and FIG. 9B.

FIGS. 10A-10C show effects of sustained treatment with UCP1 or PRDM16 (TNT) on body composition in obese mice. (FIG. 10A) Body weight, (FIG. 10B) % fat mass, and (FIG. 10C) % lean mass after 8 wks of TNT. Data are mean±S.E.M. N=6/group. *P<0.05 vs. Sham; ***P<0.001 vs. Sham.

FIGS. 11A-11C show effects of sustained treatment with UCP1 or PRDM16 (TNT) on metabolism in obese mice. (FIG. 11A) Glucose tolerance test excursion curve, (FIG. 11B) glucose tolerance test area under the curve and (FIG. 11C) fasting insulin after 6 wks of TNT. Data are mean±S.E.M. N=6/group. *P<0.05 vs. Sham.

FIGS. 12A-12F show six weeks of TNT with Prdm16 in high-fat fed mice increased cardiac function and remodeling. Data are mean±S.E.M. N=5-6 per group. *P<0.05; **P<0.01; ***P<0.001; ****P<0.0001.

FIG. 13 shows that six weeks of TNT with Ephx1/2 in high-fat fed mice resulted in gross morphological changes in the liver. Data are mean+S.E.M. N=5-6 per group.

FIGS. 14A-14D show effects of sustained treatment with 12,13-diHOME (TNT) on gene expression in obese mice. (FIG. 14A) scWAT, (FIG. 14B) pgWAT, (FIG. 14C) tibialis anterior, and (FIG. 14D) liver after 6 wks of TNT. Data are mean±S.E.M. N=6/group. *P<0.05 vs. Sham; **P<0.01 vs. Sham.

FIGS. 15A-15D show effects of sustained treatment with UCP1 or PRDM16 (TNT) on gene expression in obese mice. (FIG. 15A) scWAT, (FIG. 15B) pgWAT, (FIG. 15C) tibialis anterior, and (FIG. 15D) liver after 6 wks of TNT. Data are mean±S.E.M. N=6/group. *P<0.05 vs. Sham; **P<0.01 vs. Sham; $$P<0.01 vs. TNT-UCP1.

FIGS. 16A-16D show that TNT ALOX12 and ALOX15 improves metabolic health and cardiac function in mice. (FIG. 16A) Glucose tolerance test excursion curve and (FIG. 16B) glucose tolerance test area under the curve (GTT AUC), (FIG. 16C) ejection fraction, and (FIG. 16D) left ventricular mass after 6 weeks of TNT. Data are mean±S.E.M. N=6/group. *P<0.05; **P<0.01 vs. Sham.

### DETAILED DESCRIPTION

Disclosed herein are compositions and methods to modulate the expression of enzymes (e.g., Ephx1 and/or Ephx2) to regulate the levels of lipokines in a subject. The present disclosure provides methods for increasing levels of lipokines in a subject, comprising administering to the subject an effective amount of an epoxide hydrolase (Ephx) polypeptide or a polynucleotide encoding the Ephx polypeptide. The present disclosure also describes compositions and methods for treating a cardiovascular disease and/or an inflammatory disease comprising administering to a subject a therapeutically effective amount of an epoxide hydrolase (Ephx) polypeptide or a polynucleotide that encodes the Ephx polypeptide.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The term “biocompatible” generally refers to a material and any metabolites or degradation products thereof that are generally non-toxic to the recipient and do not cause significant adverse effects to the subject.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

A “control” is an alternative subject or sample used in an experiment for comparison purposes. A control can be “positive” or “negative.”

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

As used herein, the term “effective amount” refers to an amount of a composition necessary or sufficient to realize a desired biologic effect. An effective amount of the composition would be the amount that achieves a selected result, and such an amount could be determined as a matter of routine experimentation by a person skilled in the art. For example, an effective amount of the composition could be that amount necessary for preventing, treating and/or ameliorating the disorder described herein in a subject or could that amount necessary for increasing a level of a lipokine in a subject. The term is also synonymous with “sufficient amount.”

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g., promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “recombinant” as used herein in the context of proteins or nucleic acids refers to proteins or nucleic acids that do not occur in nature, but are the product of human engineering.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutically effective amount” or “therapeutically effective dose” of a composition (e.g., a fusion protein, a nucleic acid, or virus) refers to an amount that is effective to achieve a desired therapeutic result. In some embodiments, a desired therapeutic result is the prevention of an inflammatory disease or a cardiovascular disease. In some embodiments, a desired therapeutic result is the treatment of an inflammatory disease or a cardiovascular disease. In some embodiments, a desired therapeutic result is an increased level of a lipokine in a subject. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as coughing relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, lentiviral vectors, adenoviral vectors, adeno-associated virus vectors, retroviral vectors, and the like.

An “adeno-associated virus” or “AAV” is a virus which infects humans and some other primate species. The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1, VP2, and VP3 (molecular masses of approximately 87, 73, and 62 kDa respectively) in a ratio of about 1:1:10.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers.

Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Aci Res. 25:3389-3402, and Altschul et al. (1990) J. Mol. Biol. 215:403-410, respectively. Software for performing BLAST analyses is publicly available through the National Center for Biotechnology Information (ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for the avoidance of any doubt, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Compositions and Methods

In some aspects, disclosed herein is a composition for increasing a level of a lipokine in a subject in need thereof. In some embodiments, the composition comprises an epoxide hydrolase (Ephx) polypeptide or a polynucleotide encoding the Ephx polypeptide. A lipokine refers to a lipid-controlling hormone. It is intimately connected to intracellular pathways of fatty acid metabolism and therefore poised to communicate the intracellular energy status of adipocytes to other nonadipose tissues including liver, muscle, and pancreas. Examples of lipokines include palmitoleate, 12,13-diHOME, or fatty acid hydroxy fatty acids (FAHFA).

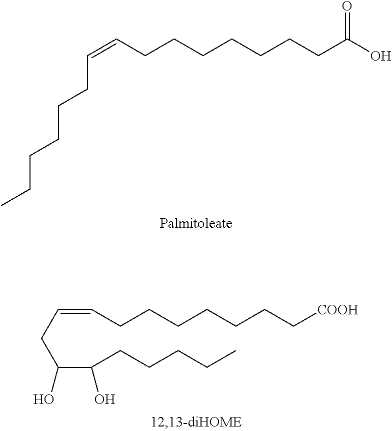 [SMILES: CCCCCC/C=C\CCCCCCCC(=O)O ; CCCCCC(O)C(O)C/C=C\CCCCCCCC(=O)O]

It should be understood that epoxide hydrolases (Ephx) are important enzymes for the biosynthesis of 12,13-diHOME. These enzymes can catalyze the conversion of 12,13-epOME to 12,13-diHOME. In some embodiments, the Ephx is Ephx1, Ephx2, Ephx3, or Ephx4. Accordingly, in some embodiments, the composition comprises an epoxide hydrolase (Ephx) polypeptide, wherein the Ephx polypeptide comprises an Ephx1 polypeptide, an Ephx2 polypeptide, an Ephx3 polypeptide, an Ephx4 polypeptide, or a combination thereof.

“Ephx1” refers herein to a polypeptide that, in humans, is encoded by the EPHX1 gene. In some embodiments, the EPHX1 polypeptide is that identified in one or more publicly available databases as follows: HGNC: 3401, NCBI Entrez Gene: 2052, Ensembl: ENSG00000143819, OMIM®: 132810, UniProtKB/Swiss-Prot: P07099. In some embodiments, the Ephx1 polypeptide comprises the sequence of SEQ ID NO: 2 or 9, or a polypeptide sequence having at or greater than about 60%, about 65%, about 70% about 75%, about 80%, about 85%, about 90%, about 95%, about 98%, or about 99% homology with SEQ ID NO: 2 or 9, or a polypeptide comprising a portion of SEQ ID NO: 2 or 9 that is a functional fragment of Ephx1. The Ephx1 polypeptide of SEQ ID NO: 2 or 9 may represent an immature or pre-processed form of mature Ephx1, and accordingly, included herein are mature or processed portions of the Ephx1 polypeptide in SEQ ID NO: 2 or 9. In some embodiments, the Ephx1 polynucleotide comprises a sequence encoding the Ephx1 polypeptide disclosed herein. In some embodiments, the Ephx1 polynucleotide comprises the sequence of SEQ ID NO: 1, or a polynucleotide sequence having at or greater than about 80%, about 85%, about 90%, about 95%, or about 98% homology with SEQ ID NO: 1, or a polynucleotide comprising a portion of SEQ ID NO: 1.

“Ephx2” refers herein to a polypeptide that, in humans, is encoded by the EPHX2 gene. In some embodiments, the EPHX2 polypeptide is that identified in one or more publicly available databases as follows: HGNC: 3402, NCBI Entrez Gene: 2053, Ensembl: ENSG00000120915, OMIM®: 132811, UniProtKB/Swiss-Prot: P34913. In some embodiments, the Ephx2 polypeptide comprises the sequence of SEQ ID NO: 4 or 10, or a polypeptide sequence having at or greater than about 60%, about 65%, about 70% about 75%, about 80%, about 85%, about 90%, about 95%, about 98%, or about 99% homology with SEQ ID NO: XX, or a polypeptide comprising a portion of SEQ ID NO: 4 or 10 that is a functional fragment of Ephx2. The Ephx2 polypeptide of SEQ ID NO: 4 or 10 may represent an immature or pre-processed form of mature Ephx2, and accordingly, included herein are mature or processed portions of the Ephx2 polypeptide in SEQ ID NO: 4 or 10. In some embodiments, the Ephx2 polynucleotide comprises a sequence encoding the Ephx2 polypeptide disclosed herein. In some embodiments, the Ephx2 polynucleotide comprises the sequence of SEQ ID NO: 3, or a polynucleotide sequence having at or greater than about 80%, about 85%, about 90%, about 95%, or about 98% homology with SEQ ID NO: 3, or a polynucleotide comprising a portion of SEQ ID NO: 3.

“Ephx3” refers herein to a polypeptide that, in humans, is encoded by the EPHX3 gene. In some embodiments, the EPHX3 polypeptide is that identified in one or more publicly available databases as follows: HGNC: 23760, NCBI Entrez Gene: 79852, Ensembl: ENSG00000105131, OMIM®: 617400, UniProtKB/Swiss-Prot: Q9H6B9. In some embodiments, the Ephx3 polypeptide comprises the sequence of SEQ ID NO: 6 or 11, or a polypeptide sequence having at or greater than about 60%, about 65%, about 70% about 75%, about 80%, about 85%, about 90%, about 95%, about 98%, or about 99% homology with SEQ ID NO: 6 or 11, or a polypeptide comprising a portion of SEQ ID NO: 6 or 11 that is a functional fragment of Ephx3. The Ephx3 polypeptide of SEQ ID NO: 6 or 11 may represent an immature or pre-processed form of mature Ephx3, and accordingly, included herein are mature or processed portions of the Ephx3 polypeptide in SEQ ID NO: 6 or 11. In some embodiments, the Ephx3 polynucleotide comprises a sequence encoding the Ephx3 polypeptide disclosed herein. In some embodiments, the Ephx3 polynucleotide comprises the sequence of SEQ ID NO: 5, or a polynucleotide sequence having at or greater than about 80%, about 85%, about 90%, about 95%, or about 98% homology with SEQ ID NO: 5, or a polynucleotide comprising a portion of SEQ ID NO: 5.

“Ephx4” refers herein to a polypeptide that, in humans, is encoded by the EPHX4 gene. In some embodiments, the EPHX4 polypeptide is that identified in one or more publicly available databases as follows: HGNC: 23758, NCBI Entrez Gene: 253152, Ensembl: ENSG00000172031, OMIM®: 617401, UniProtKB/Swiss-Prot: Q8IUS5. In some embodiments, the Ephx4 polypeptide comprises the sequence of SEQ ID NO: 8 or 12, or a polypeptide sequence having at or greater than about 60%, about 65%, about 70% about 75%, about 80%, about 85%, about 90%, about 95%, about 98%, or about 99% homology with SEQ ID NO: 8 or 12, or a polypeptide comprising a portion of SEQ ID NO: 8 or 12 that is a functional fragment of Ephx4. The Ephx4 polypeptide of SEQ ID NO: 8 or 12 may represent an immature or pre-processed form of mature Ephx4, and accordingly, included herein are mature or processed portions of the Ephx4 polypeptide in SEQ ID NO: 8 or 12. In some embodiments, the Ephx4 polynucleotide comprises a sequence encoding the Ephx4 polypeptide disclosed herein. In some embodiments, the Ephx4 polynucleotide comprises the sequence of SEQ ID NO. 7, or a polynucleotide sequence having at or greater than about 80%, about 85%, about 90%, about 95%, or about 98% homology with SEQ ID NO: 7, or a polynucleotide comprising a portion of SEQ ID NO: 7.

In some embodiments, the composition described herein comprises an Ephx1 polynucleotide. In some embodiments, the composition used herein comprises an Ephx2 polynucleotide. In some embodiments, the composition described herein comprises an Ephx3 polynucleotide. In some embodiments, the composition described herein comprises an Ephx4 polynucleotide. In some embodiments, the composition described herein comprises one or more of Ephx1, Ephx2, Ephx3, and Ephx4. In some embodiments, the composition used herein comprises an Ephx1 polynucleotide and an Ephx2 polynucleotide. In some embodiments, the polynucleotide comprises a nucleic acid sequence at least about 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1, 3, 5, or 7 or a fragment thereof.

The methods used herein for administering polynucleotide can be any transfection technologies and/or gene editing technologies known in the art, including, but not limited to AAVs, lentiviruses, electroporation, tissue nano-transfection, gene gun, or CRISPR/Cas9.

The polynucleotide disclosed herein can be contained in a vector that can be used to deliver the polynucleotide to cells, either in vitro or in vivo. The vectors and the delivery methods can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed polynucleotides (e.g., a polynucleotide encoding an Ephx1 polypeptide, a polynucleotide encoding an Ephx2 polypeptide, or a polynucleotide encoding an Ephx1 polypeptide and/or an Ephx2 polypeptide) into the cell without degradation and include a promoter yielding expression of the gene in the cells into which it is delivered. In some embodiments, the polypeptides are derived from either a virus or a retrovirus. Viral vectors can be, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. A preferred embodiment is a viral vector which has been engineered so as to suppress the immune response of the host organism, elicited by the viral antigens.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsulation, and promoters to control the transcription and replication of the viral genome. When engineered as vectors, viruses typically have one or more of the early genes removed and a gene or gene/promotor cassette is inserted into the viral genome in place of the removed viral DNA.

In some embodiments, the polynucleotide disclosed herein is contained in an adeno-associated virus (AAV) vector. This defective parvovirus is a preferred vector because it can infect many cell types and is nonpathogenic to humans. The AAV vector can further comprise the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically, the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity. The inserted genes in viral and retroviral can contain promoters, and/or enhancers to help control the expression of the desired gene product.

The AAV used herein can be an AAV serotype AAV-5, AAV-6, AAV-8 or AAV-9; a rhesus-derived AAV, or the rhesus-derived AAV AAVrh.10hCLN2; an organ-tropic AAV, or a neurotropic AAV; and/or an AAV capsid mutant or AAV hybrid serotype. In alternative embodiments, the AAV is engineered to increase efficiency in targeting a specific cell type that is non-permissive to a wild type (wt) AAV and/or to improve efficacy in infecting only a cell type of interest. It is well known in the art how to engineer an adeno-associated virus (AAV) capsid in order to increase efficiency in targeting specific cell types that are non-permissive to wild type (wt) viruses and to improve efficacy in infecting only the cell type of interest; see e.g., Wu et al., Mol. Ther. 2006 September; 14(3):316-27. Epub 2006 Jul. 7; Choi, et al., Curr. Gene Ther. 2005 June; 5(3):299-310.

In some embodiments, the composition disclosed herein is contained in or conjugated to a pharmaceutically acceptable carrier to deliver the compositions to brown adipose tissue.

In some embodiments, the composition described herein comprises an Ephx1 polypeptide. In some embodiments, the composition used herein comprises an Ephx2 polypeptide. In some embodiments, the composition used herein comprises an Ephx1 polypeptide and an Ephx2 polypeptide.

It is understood that there are numerous amino acid and peptide analogs which can be incorporated into the disclosed compositions. Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

In some embodiments, the compositions described herein are contained in or conjugated to a pharmaceutically acceptable carrier. In some embodiments, the pharmaceutically acceptable carrier is a nanoparticle. The nanoparticle used herein can be any nanoparticle useful for the delivery of polynucleotides or polypeptides. The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. In some embodiments, the nanoparticle comprises a lipid-like nanoparticle. See, for example, WO/2016/187531A1, WO/2017/176974, WO/2019/027999, or Li, B et al. Nanoparticles disclosed herein include one, two, three or more biocompatible and/or biodegradable polymers. For example, a contemplated nanoparticle may include about 10 to about 99 weight percent of a one or more block co-polymers that include a biodegradable polymer and polyethylene glycol, and about 0 to about 50 weight percent of a biodegradable homopolymer. Polymers can include, for example, both biostable and biodegradable polymers, such as microcrystalline cellulose, hydroxypropyl cellulose, hydroxypropyl methylcellulose, polyalkylene oxides such as polyethylene oxide (PEG), polyanhydrides, poly(ester anhydrides), polyhydroxy acids such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof.

In some embodiments, the nanoparticle has a diameter from about 1 nm to about 1000 nm. In some embodiments, the nanoparticle has a diameter less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm, about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a diameter, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm.

Methods

In some aspects, disclosed herein is a method for treating and/or preventing a cardiovascular disease, comprising administering to a subject a therapeutically effective amount of an epoxide hydrolase (Ephx) polypeptide or a polynucleotide that encodes the Ephx polypeptide.

In some embodiments, the composition comprises an epoxide hydrolase (Ephx) polypeptide, wherein the Ephx polypeptide comprises an Ephx1 polypeptide, an Ephx2 polypeptide, an Ephx3 polypeptide, an Ephx4 polypeptide, or a combination thereof. In some embodiments, the Ephx polypeptide comprises an Ephx1 polypeptide or an Ephx2 polypeptide. In some embodiments, the Ephx polypeptide comprises an Ephx1 polypeptide and an Ephx2 polypeptide. In some embodiments, the polynucleotide comprises a nucleic acid sequence at least about 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1, 3, 5, or 7 or a fragment thereof.

In some embodiments, the Ephx polypeptide comprises an amino acid sequence at least about 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO:2, SEQ ID NO:4, SEQ ID NO:6, SEQ ID NO:8, SEQ ID NO:9, SEQ ID NO:10, SEQ ID NO: 11, or SEQ ID NO:12.

The methods described herein providing increased levels of lipokines (e.g., using tissue nanotransfection) can negate the deleterious effects of a high-fat diet on cardiac function and remodeling, and acute injection of lipokines (e.g., 12,13-diHOME) increased cardiac hemodynamics via direct effects on the cardiomyocyte. Accordingly, in some aspects, disclosed herein are methods of treating a cardiovascular disease, comprising administering to the subject a therapeutically effective amount of an epoxide hydrolase (Ephx) polypeptide or a polynucleotide that encodes the Ephx polypeptide. In some embodiments, the cardiovascular diseases described herein are the diseases related obesity, including, for example, coronary artery disease, hypertension, stroke, atherosclerosis, coronary artery disease, heart failure, or cardiac arrhythmias.

The method disclosed herein can treat, decrease, mitigate, and/or prevent a cardiovascular disease and/or a symptom thereof (e.g., decreased systolic function, decreased diastolic function, increased posterior wall thickness, increased left ventricular mass, increased chamber dilation, and/or end diastolic volume). It should be understood and herein contemplated that the extent of effect of treating, decreasing, mitigating, and/or preventing a cardiovascular disease and/or a symptom thereof is relative to a control (e.g., a subject not being administered with the composition).

It should also be understood that obesity, which is a feature of metabolic syndrome, is associated with chronic inflammation in obese subjects. The methods and compositions disclosed herein can be used for treating and/or preventing an inflammatory disease. Accordingly, in some aspects, disclosed herein are methods of treating an inflammatory disease, comprising administering to the subject a therapeutically effective amount of an epoxide hydrolase (Ephx) polypeptide or a polynucleotide that encodes the Ephx polypeptide. In some embodiments, the inflammatory disease is type 2 diabetes or nonalcoholic fatty liver disease.

In some embodiments, the polynucleotide is contained in a vector. In some embodiments, the vector is a viral vector. In some embodiments, the viral vector is an adeno-associated virus (AAV) vector. In some embodiments, the polynucleotide is an mRNA. In some embodiments, the mRNA is contained in a nanoparticle.

In some embodiments, a level of a lipokine is increased in comparison to a reference control. The term “reference control” refers to a level in detected in a subject in general or a study population (e.g., subjects not receiving the compositions disclosed herein).

It is understood and herein contemplated that the timing of a cardiovascular disease or an inflammatory disease onset can often not be predicted. The disclosed methods of treating, preventing, reducing, and/or inhibiting a cardiovascular disease or an inflammatory disease can be used prior to or following the onset of a cardiovascular disease or an inflammatory disease. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2, or 1 hour prior to onset of a cardiovascular disease or an inflammatory disease; or 1, 2, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days, 3, 4, 5, 6, 7, 8, 9, 10, 11, 12 months, 2, 3, 4, 5, 6, 7, 8, 9, 10, 11, 12, 15, 18, 24, 30, 36, 48, 60 or more years after onset of a cardiovascular disease or an inflammatory disease.

The compositions described herein may be in any appropriate dosage form. The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intravenous, intrathecal, intravitreal, intracerebral, gingival, subgingival, intracerebroventricular, and intradermal. Such formulations may be prepared by any method known in the art. In some embodiments, the compositions disclosed herein are applied via subcutaneous route.

### EXAMPLES

The following examples are set forth below to illustrate the compounds, systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Introduction

Cardiovascular disease (CVD) is the leading cause of death across the U.S. and worldwide and affects almost half of all adults in the United States. CVD encompasses a wide range of conditions that affect the heart and vasculature including arrhythmias, dilated, hypertrophic, or idiopathic cardiomyopathies, heart failure and atherosclerosis, which can lead to fatal cardiac events such as stroke, myocardial infarction, or cardiac arrest. CVD can arise in response to multiple factors, including obesity. Obesity is an independent risk factor for the development of CVD and elevates the risk of CVD by increasing the development and severity of comorbidities such as hypertension, dyslipidemia, and diabetes.

An important therapeutic tool to combat CVD, obesity, and type 2 diabetes is exercise. Exercise remodels the heart into an “athlete's” heart, which includes physiological hypertrophy and enhanced systolic and diastolic function. This remodeling is protective to the heart, prevents the onset and development of CVD, and reflects direct modifications of the cardiomyocyte. The previous work has indicated that nitric oxide (NO) via NO synthase type 1 (NOS1) is essential for the exercise-induced effects on the cardiomyocyte.

Brown adipose tissue (BAT) is an important therapeutic tool to combat obesity and type 2 diabetes. In response to BAT transplantation, cold exposure, or exercise, BAT acts in an endocrine manner to affect whole-body metabolism and function. BAT releases ‘batokines’ including proteins and lipokines which improve glucose and fatty acid metabolism. In response to exercise and cold exposure, BAT releases the lipokine 12,13-diHOME, an oxidized linoleic acid metabolite. 12,13-diHOME acts in an autocrine and endocrine manner to increase fatty acid uptake into both BAT and skeletal muscle, reduces circulating triglycerides, and is negatively correlated with adiposity and insulin resistance in humans. Studies have indicated that BAT activity is increased in CVD and that this can have a protective effect, however no studies have investigated if BAT directly mediates cardiac function.

In this example, the inventors have identified a direct role for BAT on cardiac function mediated via 12,13-diHOME. Sustained upregulation of 12,13-diHOME by tissue nanotransfection (TNT) negated the adverse effects of a high-fat diet on cardiac function and remodeling, identifying this molecule as a therapeutic agent. Acute treatment with 12,13-diHOME increased cardiac hemodynamics via direct effects on the cardiomyocyte. Furthermore, incubation of cardiomyocytes with 12,13-diHOME increased mitochondrial respiration, and these effects were absent in NOS1^{-/-} mice and cardiomyocytes, providing a new mechanism of action for 12,13-diHOME and NOS1. This study further identified a role for 12,13-diHOME in human patients by determining that 12,13-diHOME is decreased in patients with heart disease and that this was correlated with lower ejection fraction. These results show a direct endocrine role for BAT to enhance cardiac function and, for the first time, indicate that this is mediated by regulation of calcium cycling via 12,13-diHOME and NOS1.

### Example 2. Experimental Methods

Human study protocols. The human study protocols for blood collection, assays and cardiac function data from clinical patients were approved by the AdventHealth Institutional Review Board (IRBNet #936207, 238153, and 500423) and The Ohio State University Medical Center Institutional Review Board. Study participants were recruited from The Florida Hospital Cardiovascular Institute and Transplant Institute Participants and were recruited by the study coordinator through electronic medical record (EMR) searches to identify those undergoing LVAD implantation and explanation, heart transplant, valve replacement or repair, endomyocardial biopsy during catheterization of patients with idiopathic heart failure, and arterial bypass procedures (CABG). Prior to the procedure, potential participants >18 years of age were informed about the study and if they expressed an interest, the study coordinator consented them. All patients provided written informed consent before inclusion in the study. Fasting blood samples were drawn from an antecubital vein from patients with clinical indices of heart failure (males: n=17; BMI=26.5f2.7; age=65.4f2.4; females: n=7; BMI=26.1f1.9; age=62.4f7.1) and from volunteers without heart failure (males: n=25; BMI=24.8±1.1; age=56.3±4.4; females: n=26; BMI=23.4±0.4; age=43.2±3.1). Blood was drawn into a potassium EDTA blood tube and processed per the manufacturer's instructions. Plasma aliquots were stored at −80° C. for lipidomics analysis. Ejection fraction (EF) was measured using standard cardiac magnetic resonance cine imaging, computing EF from contiguous short-axis cine images by semi-automated delineation of endocardial contours at end-systole and end-diastole (cvi42, Circle Cardiovascular Imaging, Calgary).

Mice and treatments. All animal procedures were approved by the Institutional Animal Use and Care Committee at The Ohio State University. Transplantation of brown adipose tissue (BAT) was performed as previously described using BAT removed from the intrascapular region of 12-week-old male C57BL/6 mice (Charles River Laboratories). After euthanasia of donor mice by cervical dislocation, BAT was removed and incubated in 10 ml saline at 37° C. for 20-30 minutes. Twelve-week-old C57BL/6 recipient mice were anesthetized by isoflurane inhalation in oxygen (3% isoflurane in 97% oxygen). For each recipient mouse, 0.1 g donor BAT was transplanted into the visceral cavity. The transplant was carefully lodged deep between folds within the endogenous epididymal fat of the recipient. Mice that were sham operated underwent the same procedure, but instead of receiving BAT, their epididymal fat pad was located, exposed, and then replaced.

Exercise Training Paradigm. To achieve exercise-induced adaptations on cardiac function, wild-type mice underwent an interval-based treadmill training protocol for 8 wks.

Oral gavage of sEH inhibitors. C57BL/6 male mice (Charles River Laboratories) were fed a normal chow diet before and throughout the experiment. Sham and +BAT mice underwent daily oral gavage of sEH inhibitors beginning 10 weeks after the transplant or sham surgery. Mice underwent daily oral gavage with vehicle (Phosphate Buffer Saline [PBS]), or 25 mg/L AUDA in PBS (Cayman Chemicals #10007927), or 50 mg/L t-AUCB in PBS (Cayman Chemicals #16568). Mice were gavaged at 0.5 mg/kg daily for 14-16 days.

NT device fabrication. Tissue Nano-Transfection (TNT) devices were fabricated from double side polished Silicon (Si) wafers, as reported previously. Briefly, projection lithography was used to define 400-500 nm on a photoresist. Deep reactive ion etching (DRIE) was then used drill nanochannels through the exposed Si surface. The backside of the wafers was then patterned with an array of 50 μm openings via standard photolithography followed by DRIE to gain fluidic access to the nanochannels. Finally, a 50 nm thick insulating layer of Si3N4 was deposited on the wafers via PECVD

TNT-based plasmid delivery. Six-week-old male, C57BL/6 mice were placed on a high-fat diet (60% kcal/fat) (Research Diets, Inc.) for 6 weeks prior to TNT treatment. They remained on high-fat diet throughout the TNT treatment. All plasmids (UCP1, Ephx1, Ephx2) were purchased from Origene and expanded in Escherichia coli following standard procedures. Prior to TNT, each plasmid was diluted in PBS to a final concentration of 0.05 μg/μl, and loaded into the plasmid reservoir of the TNT device. The fur was removed and the skin was exfoliated as described previously. The TNT device was then put in contact with the skin, juxtaposed to an intradermal positive electrode. The negative electrode was inserted into the plasmid reservoir, and a pulsed electric field (250 V, 10 ms pulses, 10 pulses) was applied across electrodes. Approximately 2-3 cm² were TNT-treated per mouse. This procedure was conducted directly on the skin that overlays suprascapular and inguinal BAT and WAT deposits, respectively, and was repeated weekly for a total of 8 weeks.

Lipidomic profiling and 12,13-diHOME quantification. All lipid standards were purchased from the Cayman Chemical Company. C18SPE cartridges were purchased from Biotage. All solvents are of high-performance liquid chromatography (HPLC) or LC/MS grade and were acquired from Sigma-Aldrich, Fisher Scientific or VWR International. Aliquots of 100 μl serum were used for analysis. The samples were prepared as previously described. MS analysis was performed on a SCIEX TripleTOF 6600+ system using the HR-MRM strategy consisting of a time of flight (TOF) MS experiment looped with multiple MS/MS experiments as previously described. The identity of a component was confirmed using PeakView software (SCIEX), and quantification was performed using MultiQuant software (SCIEX). The quantification of 12,13-diHOME was performed against a standard calibration curve built with fifteen points ranging from 0.01 μg/μl to 1000 μg/μl. Obtained values were corrected with the corresponding internal standard. All measurements were performed in a blinded fashion.

Measurements of Cardiomyocyte Sarcomere Function and Calcium Transient. Cardiomyocytes were isolated from wild-type C57BL/6 male mice (Charles River) or NOS1^{-/-} mice (B6; 129S4-Nos1^{tm1Plh}/J; stock no. 002633; Jackson Labs). Unloaded cardiomyocyte function (sarcomere shortening, kinetics and Ca²⁺ transients) were measured as previously performed. In brief, hearts were rapidly excised and cannulated on a constant-flow Langendorff perfusion apparatus, and perfused via the aorta at 37° C. with buffer containing (in mM) 113 NaCl, 4.7 KCl, 0.6 KH₂PO₄, 0.6 Na₂HPO₄, 1.2 MgSO₄, 12.4 BMD, 12 NaHCO₃, 10 KHCO₃, 10 HEPES 1M, and 30 Taurine, followed by digestion with liberase enzyme (0.25 mg/ml). After perfusion and digestion ventricles were removed and minced (under sterile conditions), filtered, and equilibrated with 1 mM CaCl₂ and FBS at room temperature. Cardiomyocytes were plated on laminin-coated glass slides and placed on the stage of an inverted Olympus IX-71 microscope and superfused (˜1 ml/min at 30° C.) with contractile buffer containing (in mM, pH 7.4) 4 KCl, 131 NaCl, 1 MgCl₂, 10 HEPES, 1 CaCl₂), and 10 glucose. The cells were visualized using a 40× objective and field-stimulated at 1 Hz for 3 ms using a Myopacer Field-Stimulator system. Sarcomere length, time to contraction/relaxation and contraction/relaxation kinetics will were recorded using the IonOptix video imaging system and a Myocam-S Digital charge-coupled device camera. Changes in intracellular Ca²⁺ levels were monitored using (0.5 μM) Fura-2 dual-excitation (360/380 nm) single emission (510 nm) ratiometric imaging. Measurements were taken at baseline and with acute superfusion of 10 μM 12,13-diHOME.

Seahorse bioanalyzer. Cardiomyocytes were isolated from 12-24 week old male C57BL/6 male (Charles river) or NOS1^{-/-} mice (B6; 129S4-Nos1^{tm1Pth}/J; stock no. 002633; Jackson Labs), chow fed mice. Isolated cardiomyocytes (25,000 per well) were seeded onto laminin-coated Seahorse Plates (Agilent) according to standard protocols. Cells were treated for 1 h with 10 μM 12,13-diHOME or were untreated. The oxygen-consumption rates (OCR; indicating mitochondrial respiration) and extracellular acidification rates (ECAR; indicating glycolysis rate) were monitored in a Seahorse XF24 instrument using the standard protocol of 3-min mix, 2-min wait and 3-min measure. Carbonyl cyanide-p-trifluoromethoxy-phenyl-hydrazon (FCCP; 2 μM) was used to determine the cells maximal respiratory capacity by allowing the electron transport chain to function at its maximal rate (maximal respiratory capacity is derived by subtracting non-mitochondrial respiration from the FCCP rate). Oligomycin (a complex V inhibitor; 2 μM) was used to derive ATP-linked respiration (by subtracting the oligomycin rate from baseline cellular OCR) and proton leak respiration (by subtracting non-mitochondrial respiration from the oligomycin rate). AntimycinA/Roteone (mitochondrial inhibitors; 0.5 μM) was used to determine non-mitochondrial respiration. For mechanistic experiments, cells were treated for 1 h with tetracaine (specific RyR inhibitor; 1 mM), 2,3-butanedione monoxime (BDM; myosin-ATPase inhibitor; 10 μM) and nifedipine (L-type calcium channel inhibitor; 10 μM) were used. Data from wells of the same treatment group were averaged together and analyzed directly using Waves software. For the normalization of respiration to protein content, cells were lysed in RIPA buffer and protein concentration was measured by Bradford assay.

Cardiomyocyte Fatty Acid Uptake. Cardiomyocytes were isolated from 12-24 week old male LucTg (FVB-Tg(CAG-luc-GFP)L2G85Chco/J; stock no. 008450; Jackson Labs) chow-fed mice. Isolated cardiomyocytes (50,000 cells per well) were seeded onto laminin-coated 12-well plates according to standard protocols in FBS-free media for one hour. After one hour of serum-starve, 10 μm FFA-SS-Luc (Intrace Medical) conjugated probe was added to each well with or without 10 μM 12,13-diHOME directly before imaging using the IVIS Spectrum for fluorescent optical imaging using sequential 3 min exposures for 30 min. Data were analyzed using Living Image Software, and movies were assembled from individual images using ImageJ.

In vivo cardiac function. Wild-type C57BL/6 male mice (Charles river) or NOS1^{-/-} mice (B6; 129S4-Nos1^{tm1Pth}/J; stock no. 002633; Jackson Labs) were anesthetized with 1-2% isoflurane and echocardiography was conducted using a Vevo 2100 Ultrasound. Echocardiogram data was analyzed using VevoLab software to determine left ventricle (LV) ejection fraction, LV mass, and LV diastolic diameter.

Cardiac pressure-volume analysis. Hemodynamic, systolic, and diastolic measurements performed as previously done. Briefly, mice were anesthetized with 1% isoflurane and a 1.4 F Millar catheter was advanced from the carotid artery into the left ventricle. LV pressure-volume dynamics were simultaneously measured over a wide range of heart rates (240-600 bpm). For the TNT experiments, since there were different baseline heart rates, data was normalized to the lowest heart rate. For the acute injection experiments, 12,13-diHOME, 9,10-diHOME, or 9-HODE were infused through the femoral vein (all at 1.5 μg/kg). All pressure-volume loop parameters were calculated using PV loop analysis software module for Lab Chart on ADInstruments.

Physiological and Biochemical Methods. For intraperitoneal glucose tolerance tests (GTTs), mice were fasted for 11 h (2200 to 0900 h) with free access to drinking water. A baseline blood sample was collected from the tails of fully conscious mice, followed by intraperitoneal injection of glucose (2 g glucose/kg body weight), and blood was taken from the tails for glucose measurements at 0, 15, 30, 60, and 120 min. The assessment of fat and lean mass was performed using an Echo-MRI-3-in-1.

Comprehensive Lab Animal Monitoring System. The Comprehensive Lab Animal Monitoring System (Oxymax Opto-M3; Columbus Instruments) was used to measure activity level, volume of O₂ consumption, volume of CO₂ production, and heat production. Total energy expenditure of mice was calculated as described previously.

Statistical Analysis. The data are presented as means A SEM. Statistical significance was defined as P<0.05 and determined by one- or two-way ANOVA, or repeated measures two-way ANOVA, with Tukey and Bonferroni post hoc analysis, or unpaired two-tailed Student's t-test. For experiments with human subjects, linear regression analyses were used for analysis. Kolmogorov-Smirnov test were used for normality tests.

TABLE 1
PCR primer sequences

| | | | |
|---|---|---|---|
| 127F | Ephx1 | Mouse | CAATGGTTCCTGTCGTCCAGTAG (SEQ ID NO: 13) |
| 127R | Ephx1 | Mouse | AGTTCTCCACCTGGACCAAGTC (SEQ ID NO: 14) |
| 128F | Ephx2 | Mouse | ATCTGGTGGCATAAACGGCGTG (SEQ ID NO: 15) |
| 128R | Ephx2 | Mouse | CCCTCAAGCAGTGTTCATTGGC (SEQ ID NO: 16) |
| 129F | Ephx3 | Mouse | GGAAATGGCTCTGAAACTCTCGC (SEQ ID NO: 17) |
| 129R | Ephx3 | Mouse | GCACTATGTCTCTGCTGGTCATG (SEQ ID NO: 18) |
| 130F | Ephx4 | Mouse | GCCATCTCTACCTCCATAAACGC (SEQ ID NO: 19) |
| 130R | Ephx4 | Mouse | TCTCAGCCTGGAGCACTAAGTG (SEQ ID NO: 20) |
| 253-F | EPHX2-2 | Mouse | ACCACTCATGGATGAAAGCTACA(SEQ ID NO: 21) |
| 253-R | EPHX2-2 | Mouse | TCAGGTAGATTGGCTCCACAG (SEQ ID NO: 22) |
| 384F | EPHX1 | Human | GTTTTCCACCTGGACCAATACGG (SEQ ID NO: 23) |
| 384R | EPHX1 | Human | TGGTGCCTGTTGTCCAGTAGAG (SEQ ID NO: 24) |
| 385F | EPHX2 | Human | AGCCTCTTCAGAGCAAGCGATG (SEQ ID NO: 25) |
| 385R | EPHX2 | Human | GGATTTCCTCCTCAGTGACCATC (SEQ ID NO: 26) |
| F | EPHX3 | Human | TGTTGTGGCTGTGGACTTGCGA (SEQ ID NO: 27) |
| R | EPHX3 | Human | GCCACAAGGATGCACTTCGAGT (SEQ ID NO: 28) |
| F | EPHX4 | Human | CTGCTGGAGAAAGAGGCAAACC (SEQ ID NO: 29) |
| R | EPHX4 | Human | CCATAACCTCTCAAATCCAGTGC (SEQ ID NO: 30) |

### Example 3. Transplantation of BAT and Exercise Improve Cardiac Function in Mice

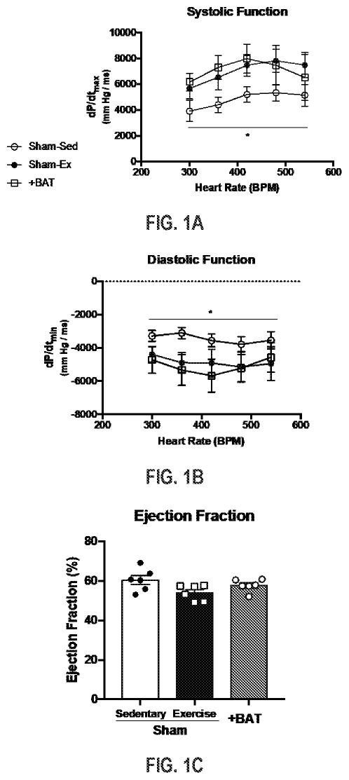

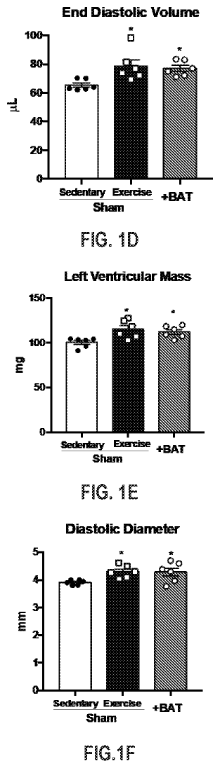

To determine the effects of increasing BAT mass by transplantation on cardiac function, mice were transplanted with 0.1 g BAT (+BAT) into the visceral cavity from age and gender matched control mice. Twelve weeks post-transplantation, in Nivo cardiac hemodynamics revealed that +BAT mice had improved systolic function (FIG. 1A). dP/dtₘᵢₙ was measured and a more negative dP/dtₘᵢₙ was determined in +BAT mice, indicating accelerated relaxation, and thus enhanced diastolic function (FIG. 1B) compared to Sham mice. +BAT did not alter ejection fraction (FIG. 1C), but resulted in beneficial cardiac remodeling (FIGS. 1D-1F).

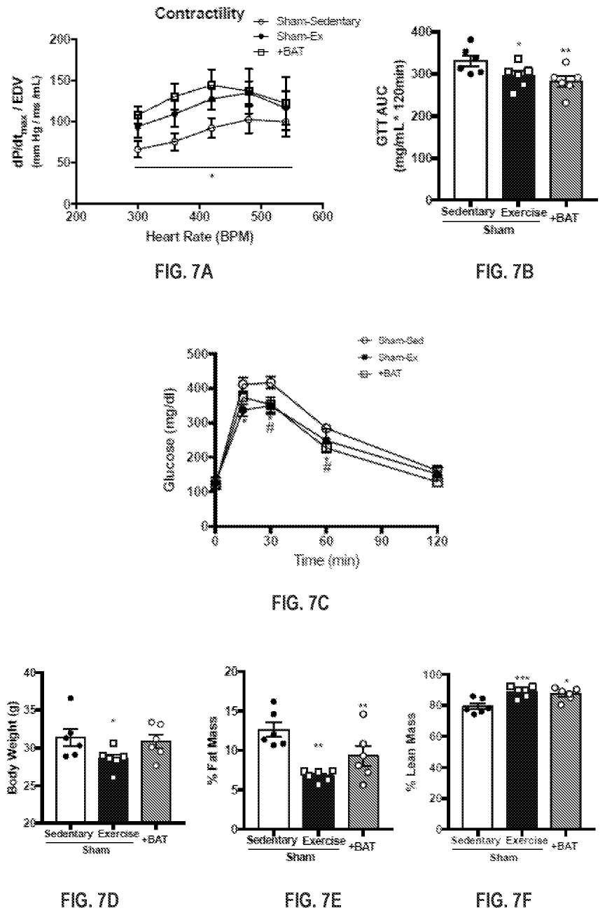

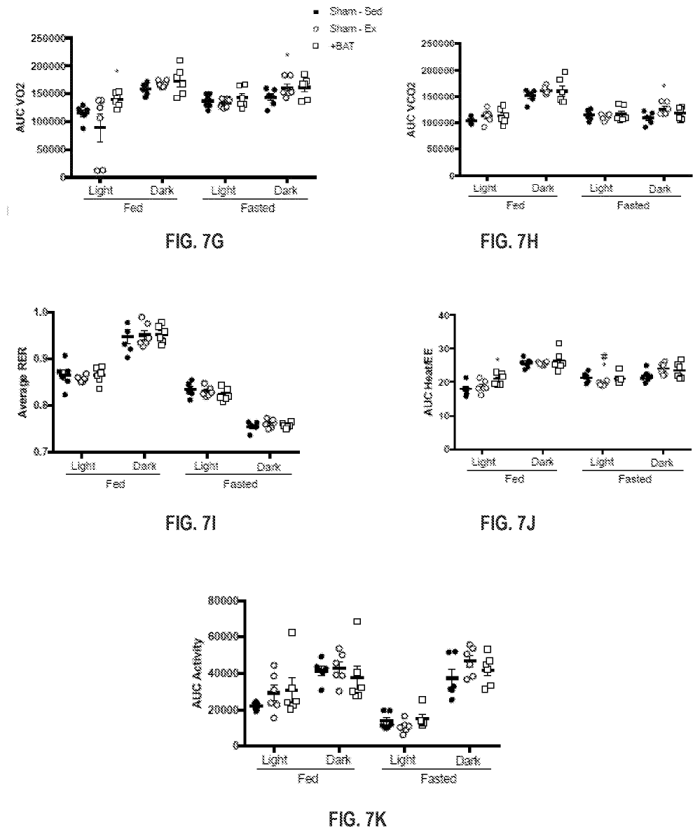

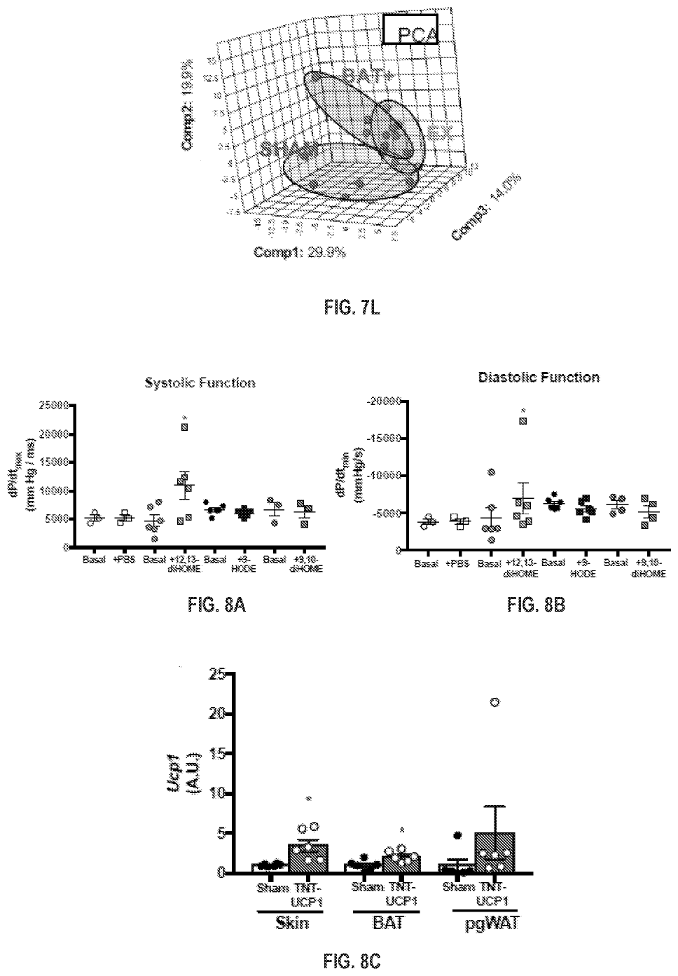

It is well-established that exercise-training influences cardiac function, and a role for exercise to influence the endocrine function of BAT has been identified. To determine if exercise and +BAT resulted in similar adaptations to cardiac function, a group of Sham-operated surgical control mice underwent 8 weeks of intense exercise interval training beginning four weeks post-surgery, and cardiac function was assessed. Both exercise and +BAT had similar improvements on in vivo cardiac hemodynamics (FIGS. 1A-1B) but no change in ejection among groups (FIG. 1C). Markers of cardiac remodeling, including end diastolic volume (EDV), left ventricular mass (LVM), and diastolic diameter were increased with both exercise and +BAT (FIGS. 1D-1F). To determine changes in contractility were also involved, the data was normalized for EDV. When normalized, an increase in contractility contributed to the enhanced systolic function (FIG. 7A). Taken together these data indicate that both exercise and +BAT have a similar effect to improve cardiac function via preload (EDV) and contractility in mice.

### Example 4. Exercise and +BAT Improve Metabolic Health

Given that +BAT improves metabolic health in mice, the effects of +BAT and exercise on glucose tolerance and body composition were examined. Metabolic testing was performed at the same time as in vivo cardiac function testing and revealed that both exercise and +BAT improved glucose tolerance (FIGS. 7B-7C). Exercise-training decreased body weight (FIG. 7D), and both exercise and +BAT decreased % fat mass and increased % lean mass (FIGS. 7E-7F). There was a minimal effect of +BAT or exercise on V_{O2}, V_{CO2}, respiratory exchange ratio (RER), heat and energy expenditure, or spontaneous activity (FIGS. 7G-7K). These data indicate that both exercise and +BAT improve glucose tolerance and decrease % fat mass to a similar extent.

### Example 5. 12,13-diHOME is Up-Regulated with +BAT and Exercise

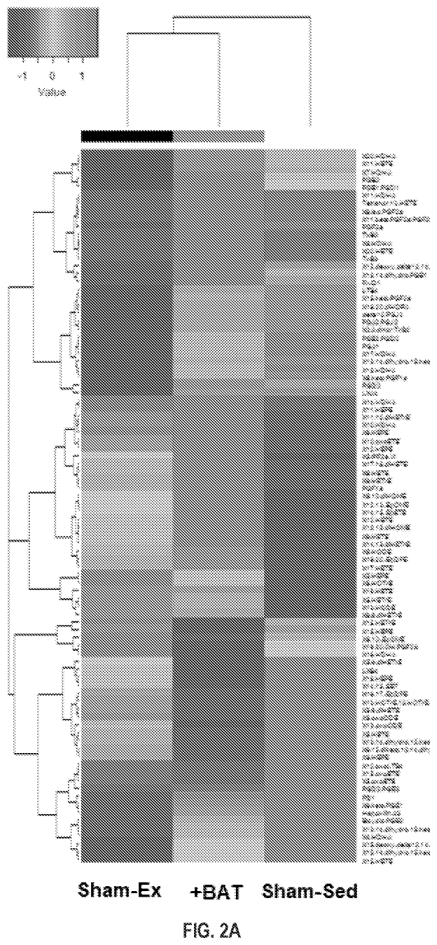

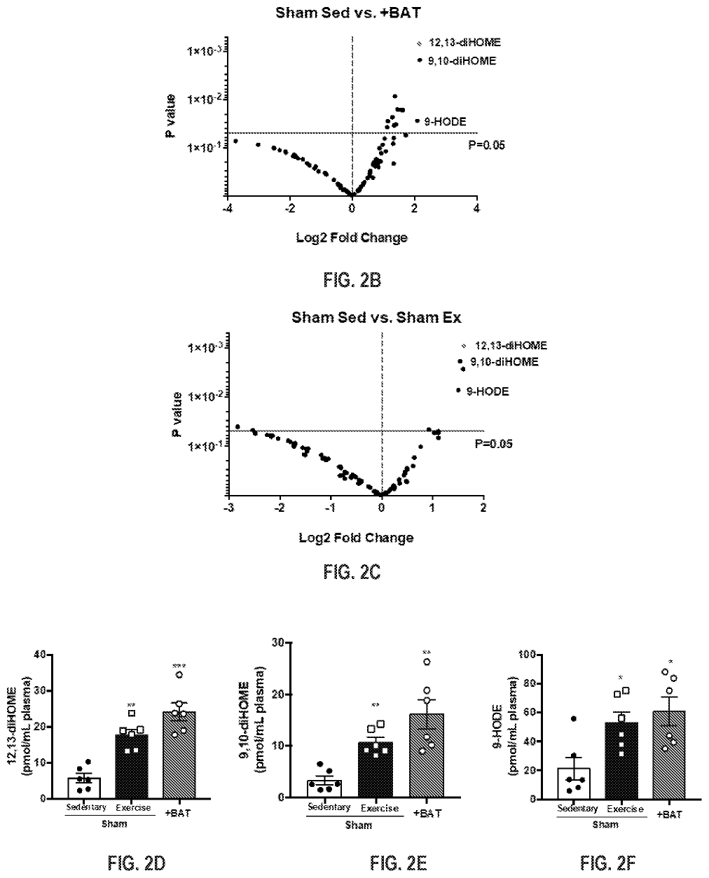

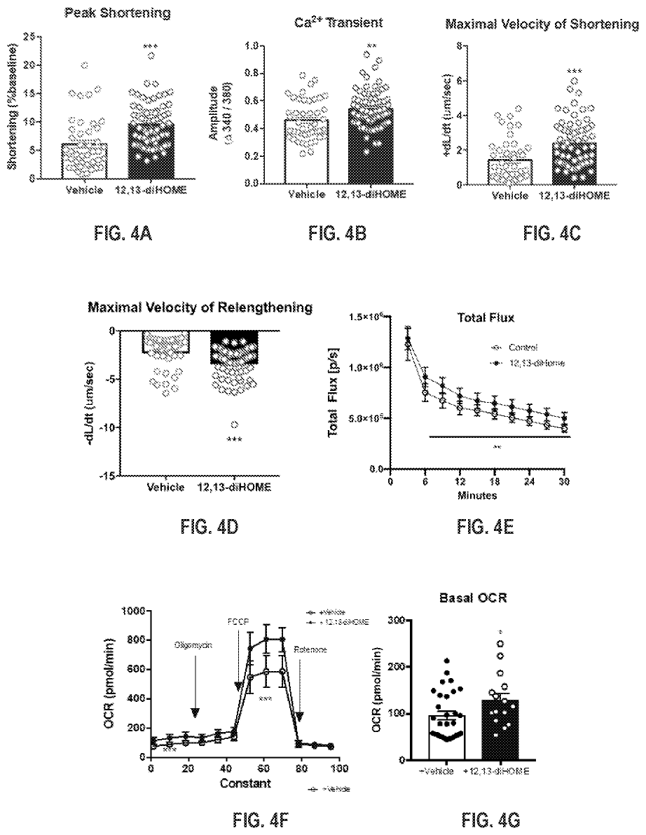

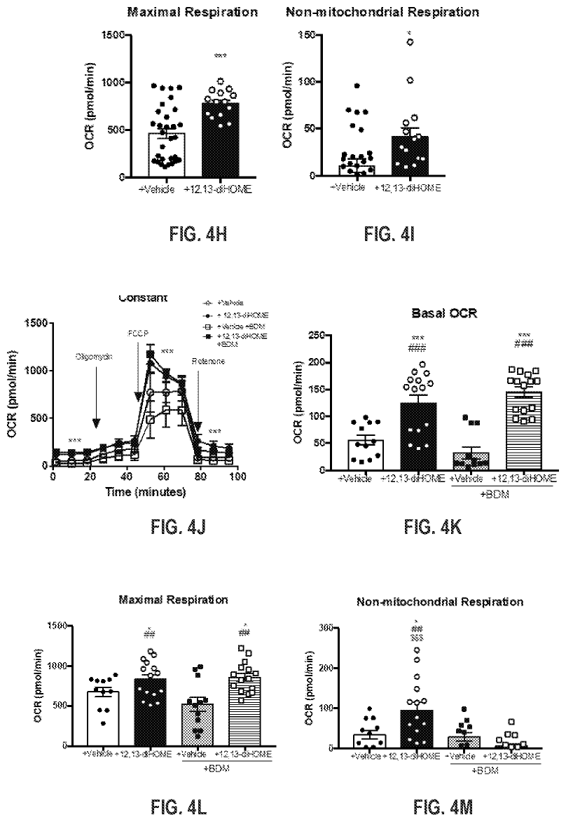

The effect of exercise and +BAT on circulating signaling lipokines was examined. Plasma was analyzed by liquid chromatography tandem mass spectrometry (LC-MS/MS) to measure the concentrations of a panel of 90 mediator lipids with annotated signaling properties (Complete lipidomics data is submitted to metabolomicsworkbench.org). Hierarchical clustering revealed similarities between +BAT and Sham-Exercised, but no clear pattern of lipokine regulation among Sham-Sedentary, +BAT, or Sham-Exercised mice (FIG. 2A; FIG. 7L). Eleven signaling lipids were increased in +BAT mice (FIG. 2B) and 4 signaling lipids were increased with exercise (FIG. 2C). Of these, there were three of the same lipokines increased among +BAT and exercise: 12,13-diHOME, 9,10-diHOME, and 9-HODE (FIGS. 2D-2F). The signaling lipid that was the most significantly increased by p-value and fold change in both the +BAT and exercised mice was 12,13-diHOME (FIGS. 2B-2D).

### Example 6. 12,13-diHOME Increases In Vivo Cardiac Hemodynamics

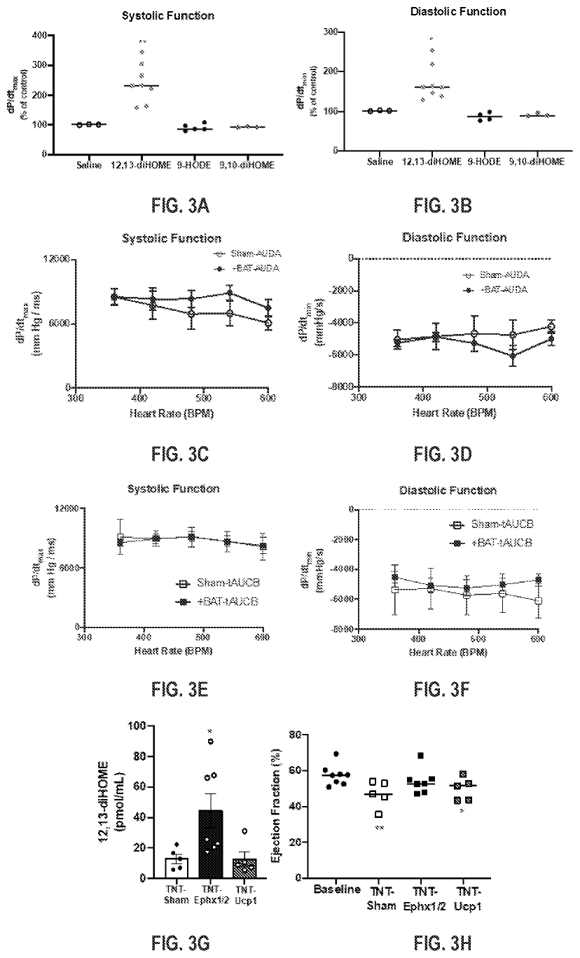

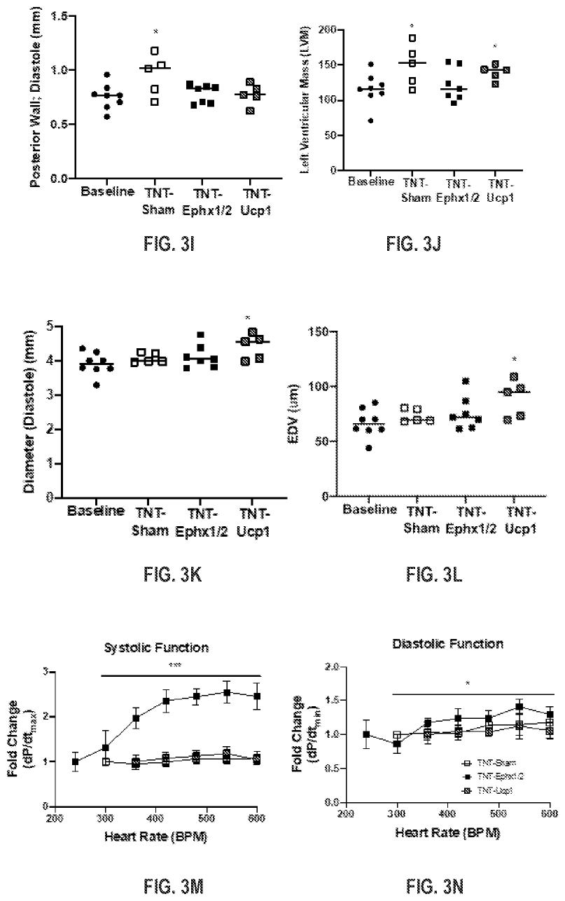

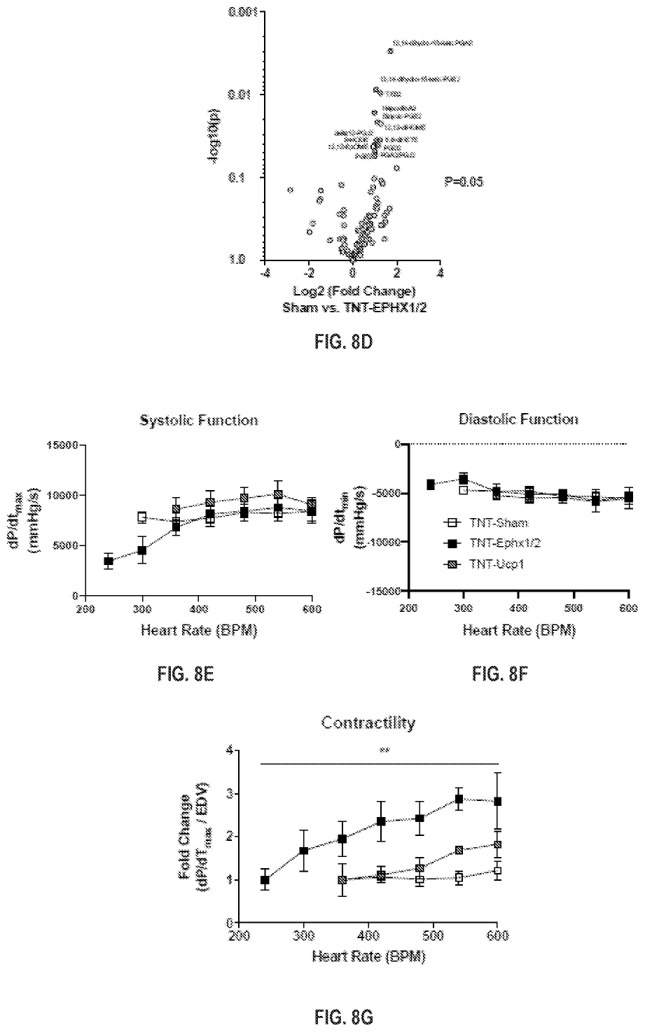

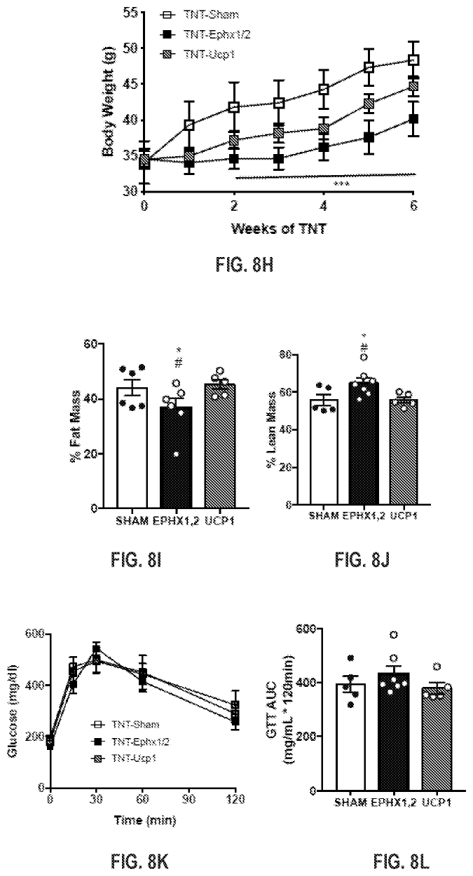

Both +BAT and exercise improved cardiac function in mice and increased circulating 12,13-diHOME. To determine if 12,13-diHOME was responsible for the improved cardiac function in +BAT and exercise-trained mice, mice were acutely injected with 12,13-diHOME and in vivo cardiac hemodynamics were measured. Acute treatment of 12,13-diHOME improved systolic function (FIG. 3A; FIG. 8A) and diastolic function (FIG. 3B; FIG. 8B) compared to mice injected with saline. There was no effect of 9,10-diHOME or 9-HODE to alter systolic (FIG. 3A; FIG. 8A) or diastolic function (FIG. 3B; FIG. 8B). These data show that 12,13-diHOME is a potent positive inotropic and lusitropic modulator in mice.

### Example 7. sEH Inhibition Negates the Improvement in Cardiac Function with BAT

After observing the effect of 12,13-diHOME to acutely increase in vivo cardiac hemodynamics, the next experiment was performed to investigate if inhibition of 12,13-diHOME prevented the BAT-induced improvements in cardiac function. To do this, mice were divided into either Sham or +BAT, and 10 weeks after surgery fed daily via oral gavage with the soluble epoxide hydrolase (sEH) inhibitors AUDA or t-AUCB for 2 weeks. sEH are the enzymes that produce 12,13-diHOME. In the presence of sEH inhibitors, there was no effect of +BAT to improve cardiac function (FIGS. 3C-3F). These data provide further evidence that the improvements in cardiac function via BAT are mediated through sEH, and 12,13-diHOME.

### Example 8. Sustained Treatment with 12,13-diHOME Negates the Deleterious Effects of a High-Fat Diet on Cardiac Structure and Function

To test the therapeutic applications of 12,13-diHOME, mice were placed on a high-fat diet for 6 wks and tissue nanotransfection (TNT) was performed using plasmids that expressed proteins of interest. These plasmids were electroplated on the skin that overlays BAT and WAT depots once a week for 8 wks to drive sustained expression of soluble epoxide hydrolase 1 and 2 (Ephx1/2; TNT-Ephx1/2) or uncoupling protein 1 (Ucp1; TNT-Ucp1). Ephx1/2 was selected because it is an important enzyme for the biosynthesis of 12,13-diHOME. 12,13-diHOME is regulated by soluble epoxide hydrolases (sEH) of which Ephx1 and Ephx2 are the major isoforms expressed in adipose tissue. After the conversion of linoleic acid by cytochrome P450 to 12,13-epOME, Ephx1/2 are soluble epoxide hydrolases (sEH) that catalyze the conversion of 12,13-epOME to 12,13-diHOME. As such, TNT-driven overexpression of Ephx1 and 2 can correlate with increased synthesis of 12,13-diHOME in overlaying skin and circulation. TNT-driven overexpression of UCP1 (TNT-Ucp1) was used as a proxy for BAT-mediated activity since UCP1 is the predominant marker of BAT and plays an important role in non-shivering thermogenesis. To confirm effectiveness of TNT-Ucp1, Ucp1 was measured in skin, BAT, and perigonadal white adipose tissue (pgWAT). Ucp1 was significantly increased in skin and BAT of TNT-Ucp1 mice (FIG. 8C). Control mice (TNT-Sham) underwent a sham procedure of weekly anesthetization only. Signaling lipids were measured (FIG. 8D) and TNT-Ephx1/2 increased 12,13-diHOME in circulation, but 12,13-diHOME was not altered in TNT-Ucp1 mice (FIG. 3G). All mice were compared to a baseline group; chow-fed, aged-matched mice, in order to account for the effects both high-fat diet and TNT. Similar to previous studies, a high-fat diet resulted in adverse cardiac remodeling as observed by a decrease in ejection fraction (FIG. 3H) and an increase in posterior wall thickness and left ventricular mass in the TNT-Sham mice (FIGS. 3I, 3J). TNT-Ucp1 mice also had significant chamber dilation and end diastolic volume (FIGS. 3K, 3L). However, TNT-Ephx1/2 mice were completely protected from the pathological remodeling induced by high-fat diet (FIGS. 3I-3L). Investigation of in vivo cardiac hemodynamics revealed that although systolic and diastolic function were not different (FIGS. 8E-8F), the TNT-Ephx1/2 mice maintained the force frequency response in systole (FIG. 3M) and diastole (FIG. 3N), indicating increased function with an increasing heart rate. The force frequency response was blunted in systole and diastole in the TNT-Sham and TNT-Ucp1 mice, consistent with what is observed in heart disease and type 2 diabetes (FIGS. 3M, 3N). To ascertain if these differences can be due to an increase in contractility, the data were normalized to EDV. Contractility was increased in TNT-Ephx1/2 mice compared to both Sham and TNT-UCP1 mice (FIG. 8G). These data indicate that an increase in TNT-Ephx1/2 negated the deleterious effects of a high-fat diet on cardiac function and remodeling. High-fat diet significantly increased body weight in the TNT-Sham and TNT-Ucp1 mice, but TNT-Ephx1/2 mice were protected from the high-fat diet-induced weight gain (FIG. 8H). TNT-Ephx1/2 mice also had decreased % fat mass, and increased % lean mass, compared to other groups (FIGS. 81 and 8J). There was no effect of increasing TNT-Ephx1/2 or TNT-Ucp1 on glucose tolerance (FIGS. 8K, 8L), similar to previous studies investigating the effects of two weeks of chronic injection of 12,13-diHOME. Together these data highlight the importance of the endocrine role for BAT to have a protective effect on cardiac structure and function, as TNT-driven modulation of Ucp1 expression resulted in adverse cardiac remodeling, while an increase in 12,13-diHOME (TNT-Ephx1/2) was protective.

### Example 9. 12,13-diHOME Increases Contractile Function and Calcium Uptake in Isolated Cardiomyocytes

After determining an increase in contractility observed in vivo, the next experiment further investigated if 12,13-diHOME had a direct effect on the function of isolated cardiomyocytes. Acute superfusion with 12,13-diHOME increased peak cardiomyocyte shortening (FIG. 4A) via an increase in Ca² transient amplitude (FIG. 4B). 12,13-diHOME also resulted in faster kinetics determined as greater maximal velocity of shortening (+dL/dt) (FIG. 4C), and maximal velocity of relengthening (−dL/dt) (FIG. 4D). These data indicate that 12,13-diHOME directly increases cardiomyocyte function via enhanced Ca²⁺ handling, consistent with the in vivo data indicating that 12,13-diHOME is a positive inotrope.

### Example 10. 12,13-diHOME Increases Fatty Acid Uptake in Cardiomyocytes

Previous studies have indicated a role for 12,13-diHOME to increase fatty acid uptake. To test whether 12,13-diHOME increases fatty acid uptake in cardiomyocytes, cardiomyocytes constitutively expressing luciferase in vitro were isolated. Cells were treated with FFA-SS Luc, a fatty acid conjugated to luciferin, in the presence of 12,13-diHOME or a vehicle control. Fatty acid uptake was significantly elevated in isolated cardiomyocytes treated with 12,13-dihOME after 30 min of incubation (FIG. 4E). These data indicate that 12,13-diHOME increases fatty acid uptake in cardiomyocytes, similar to its effect on BAT and skeletal muscle.

### Example 11. 12,13-diHOME Increases Respiration in Cardiomyocytes

Based on data indicating that 12,13-diHOME increases mitochondrial function in BAT and skeletal muscle, whether 12,13-diHOME regulated mitochondrial function in cardiomyocytes was tested. 12,13-diHOME increased basal oxygen consumption rates (OCR), maximal respiratory capacity, and non-mitochondrial respiration in isolated cardiomyocytes (FIGS. 4F-4I).

To determine if 12,13-diHOME increased mitochondrial respiration directly or indirectly via greater energy demand due to increased contraction, OCR was measured in cardiomyocytes incubated with 12,13-diHOME and the myosin inhibitor 2,3-Butanedione monoxime (BDM). Inhibition of myosin did not prevent 12,13-diHOME to increase basal OCR or maximal respiration, but prevented the increase in non-mitochondrial respiration (FIGS. 4J-4M). These data indicate that 12,13-diHOME increases respiration in cardiomyocytes.

### Example 12. Mechanism of Action of 12,13-diHOME in Cardiac Function

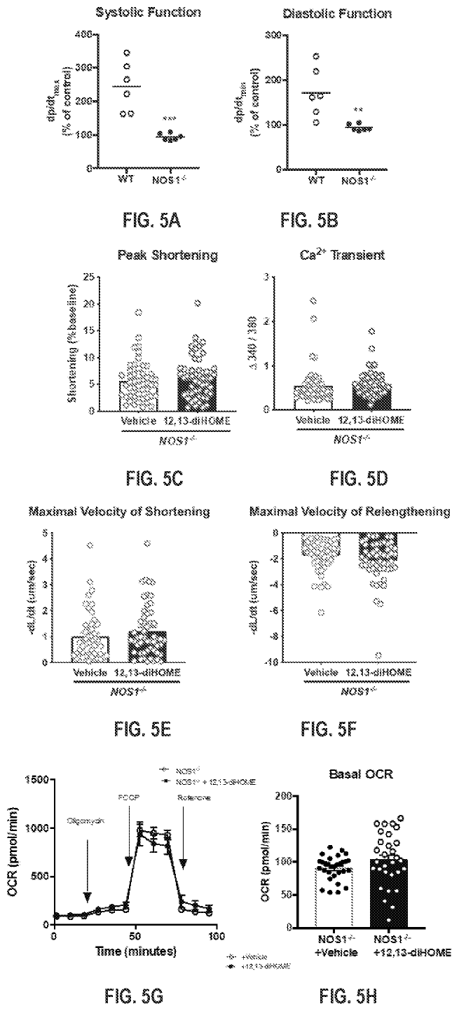

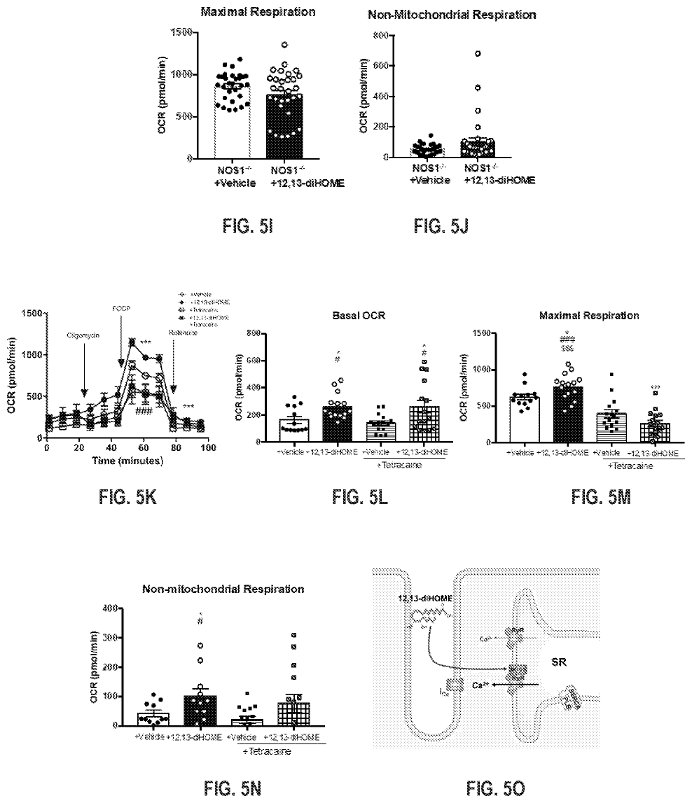

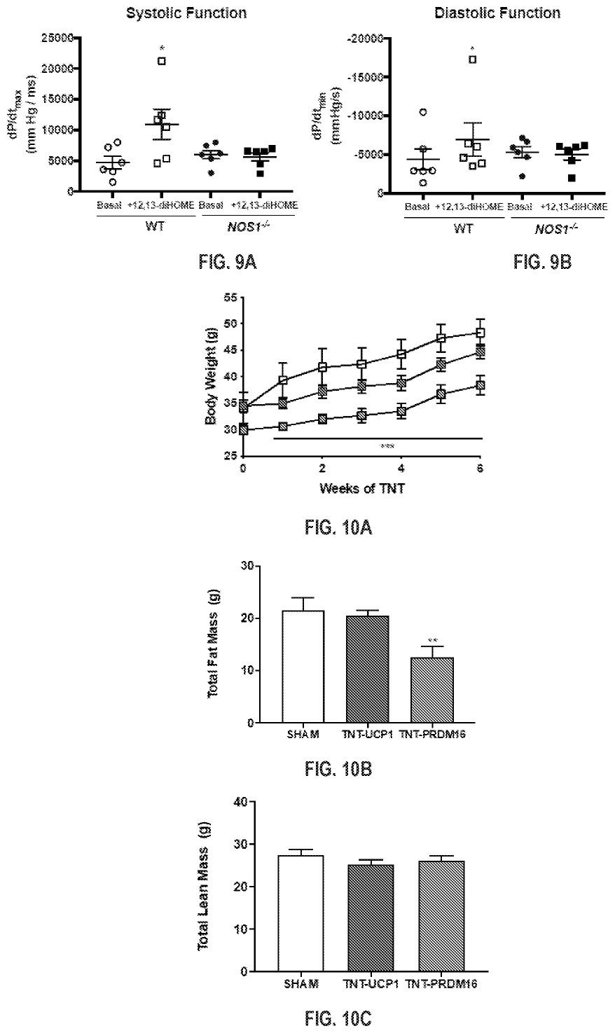

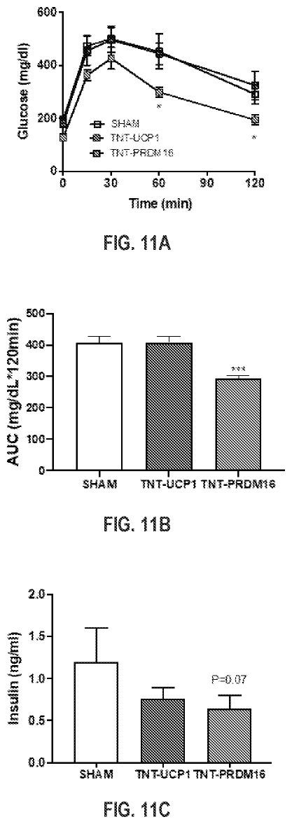

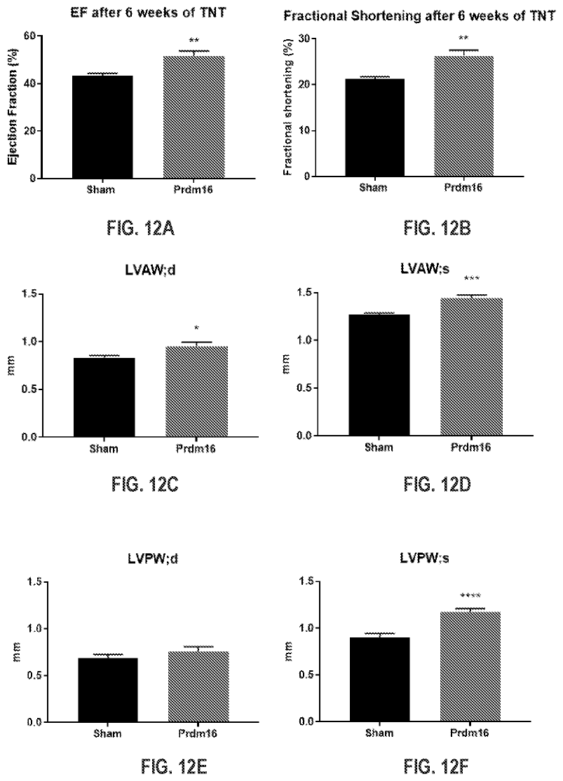

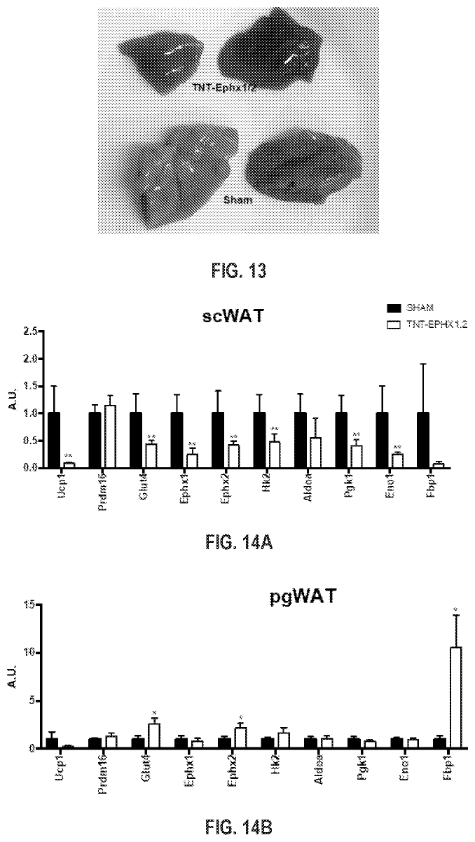

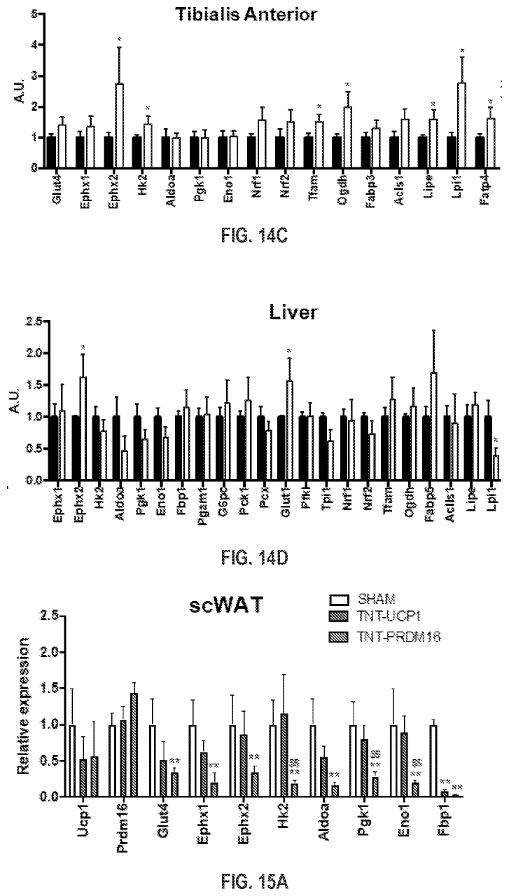

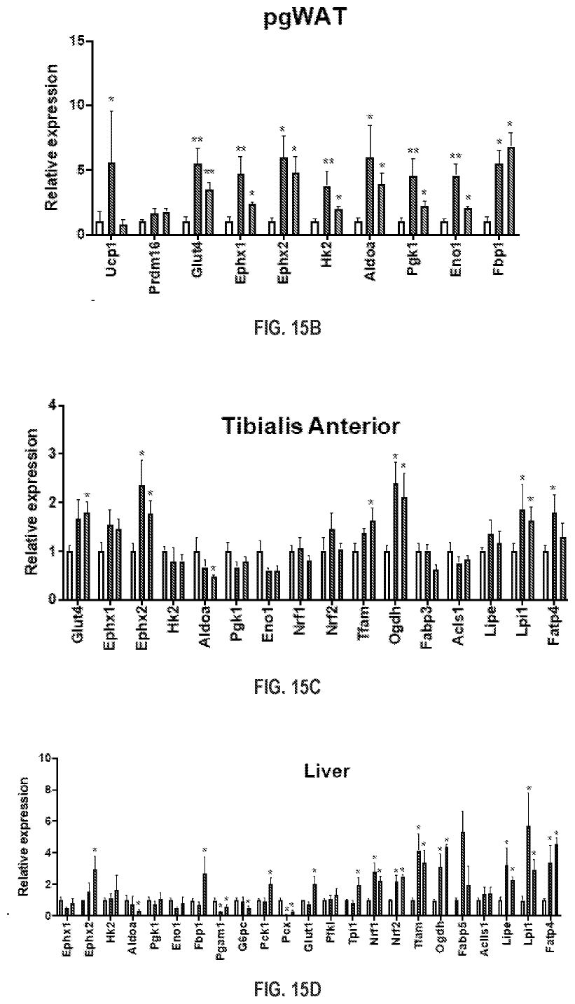

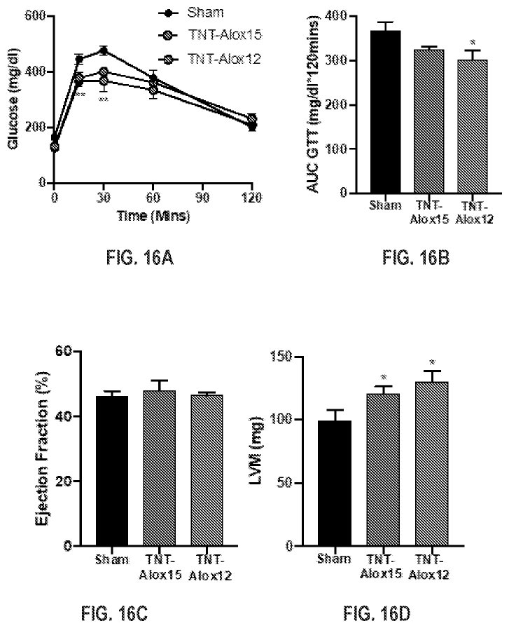

Next, the mechanism through which 12,13-diHOME modulates cardiomyocyte function and respiration was investigated. It was shown that nitric oxide (NO) production via NO synthase type 1 (NOS1) is essential for the beneficial effects of exercise to the heart. Since +BAT had similar effects on the heart as exercise, 12,13-diHOME can function through a similar mechanism. Mice deficient in NOS1 (NOS1^{-/-}) were acutely injected with 12,13-diHOME and in vivo cardiac hemodynamics were measured. In contrast to what was observed in wild-type (WT) mice (FIGS. 3A, 3B), there was no effect of acute injection of 12,13-diHOME on systolic (FIG. 5A; FIG. 9A) or diastolic (FIG. 5B; FIG. 9B) function in NOS1^{-/-} mice. In addition, 12,13-diHOME had no effect on NOS1^{-/-} cardiomyocyte peak shortening, Ca²⁺ transients, or kinetics (FIGS. 5C-5F). These data indicate that 12,13-diHOME confers beneficial effects on cardiac function via activation of NOS1.

In NOS1^{-/-} cardiomyocytes there was no effect of 12,13-diHOME to increase basal oxygen consumption rates (OCR), maximal respiratory capacity, and non-mitochondrial respiration in cardiomyocyte (FIGS. 5G-5J). Since previous work indicated that NOS1 signaling activates the ryanodine receptor (RyR), if RyR was involved in the 12,13-diHOME signaling pathway was further investigated. Cardiomyocytes isolated from wild-type mice were incubated with 12,13-diHOME and the RyR2 inhibitor, tetracaine. 12,13-diHOME increased basal OCR in the presence or absence of tetracaine (FIGS. 5K, 5L), but incubation with tetracaine blunted the effect of 12,13-diHOME on maximal respiration and non-mitochondrial respiration (FIGS. 5K, 5M, 5N). Increasing Ca²⁺ cycling within the cardiomyocyte increases mitochondrial respiration, and inhibiting RyR via tetracaine prevents the increase in Ca²⁺ cycling and thus maximal respiration. This is consistent with the decrease in maximal respiration observed in cardiomyocytes treated with tetracaine, independent of 12,13-diHOME. Thus, these data indicate that 12,13-diHOME increases maximal mitochondrial respiration, via enhanced Ca²⁺ cycling. Together these data indicate that 12,13-diHOME increases cardiomyocyte contraction and respiration via a NOS1-dependent mechanism, and cardiomyocyte contraction is likely mediated via RyR (FIG. 5O).

### Example 13. 12,13-diHOME is Decreased in Human Heart Disease Patients

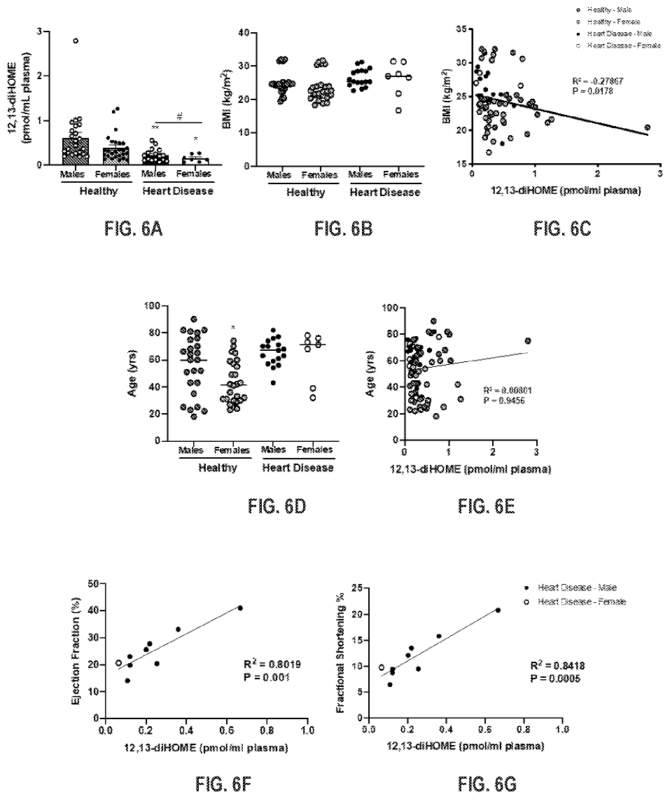

To determine if there was a correlation between 12,13-diHOME and heart disease or cardiac function in humans, 12,13-diHOME was measured in a cohort of 75 male and female subjects, with or without heart disease. This cohort consisted of healthy males (n=25; BMI=24.8±1.1; age=56.3±4.4), healthy females (n=26; BMI=23.4±0.4; age=43.2±3.1), males with heart disease (n=17; BMI=26.5±2.7; age=65.4±2.4), and females with heart disease (n=7; BMI=26.1±1.9; age=62.4±7.1). Both male and female subjects with heart disease had reduced concentrations of 12,13-diHOME (FIG. 6A). There was no difference in BMI among groups (FIG. 6B), but 12,13-diHOME was negatively correlated to BMI consistent with previous studies (FIG. 6C). There was no difference in age among groups (FIG. 6D) and age was not correlated to 12,13-diHOME in this cohort of human subjects (FIG. 6E). Functional cardiac measurements were obtained in a subset of patients with heart disease, and these data revealed that ejection fraction (FIG. 6F) and fractional shortening (FIG. 6G) were positively correlated with 12,13-diHOME, indicating an important role for 12,13-diHOME to have a protective effect on cardiac function.

Here, the study establishes a novel paradigm in which BAT plays a critical endocrine role to directly affect cardiac function and metabolism via 12,13-diHOME. 12,13-diHOME improves in vivo cardiac hemodynamics by increasing cardiomyocyte contraction, relaxation, and mitochondrial respiration. Sustained treatment with 12,13-diHOME negates the deleterious effects of a high-fat diet on cardiac function and remodeling, an effect that was not seen in TNT-Ucp1 mice. This study further identified that 12,13-diHOME mediates these beneficial effects through the activation of NOS1. Human patients with heart disease had decreased concentrations of 12,13-diHOME, and 12,13-diHOME was directly correlated to ejection fraction in these patients. These data demonstrate an important mechanism for the endocrine role of BAT to mediate cardiac function in health and disease through the release of the lipokine, 12,13-diHOME.

Elucidating mechanisms to combat obesity and its co-morbidities, including cardiovascular disease (CVD), have become an increasingly important research focus. Brown adipose tissue (BAT) has been identified as an important target to combat obesity, but its role to combat CVD had not been thoroughly investigated. Studies have indicated that BAT activity (measured as UCP1 expression) is increased in CVD in mouse models, but it is not clear if this is detrimental or protective for cardiac function. This study shows that it is the endocrine function of BAT, independent of any measurement of activity, that has a protective effect on cardiac function.

A role for the batokine, 12,13-diHOME, to mediate cardiac function was identified both in vivo and in vitro. Similar to its acute effect on BAT and skeletal muscle, 12,13-diHOME also increases mitochondrial respiration in the isolated cardiomyocyte. Thus, 12,13-diHOME directly increases generation of energy in response to its effect of enhancing the workload of the heart. These data indicates that 12,13-diHOME mediates beneficial actions on cardiac function via NOS1 within the cardiomyocyte, and these are consistent with previous studies showing that NOS1 enhances cardiac contraction via activation of RyR. Furthermore, sustained treatment with 12,13-diHOME was cardioprotective. In contrast to the data, some studies have indicated a role for 12,13-diHOME to impair cardiac health, however these studies were performed in ex vivo or ischemia/reperfusion models, or at concentrations known to be toxic to cardiomyocytes. These data indicate that at supraphysiological concentrations, 12,13-diHOME has a consistent beneficial effect on both in vivo and in vitro cardiac function and metabolism. Further, these data in human subjects indicate that 12,13-diHOME is decreased in heart disease but positively correlated with ejection fraction, showing it as a therapeutic. It is important to note that the human functional measurements were only performed in a small population of patients (n=9), only in patients with heart disease, and mostly male patients (n=8 males; n=1 female). Further studies must be performed to fully characterize the correlation of cardiac function and 12,13-diHOME in healthy subjects or other subpopulations. These data show that 12,13-diHOME can have beneficial clinical ramifications; however, more long-term studies are be needed to determine the safety and efficacy of 12,13-diHOME treatment.

In conclusion, BAT functions in an endocrine manner to directly modulate the heart via release of the lipokine 12,13-diHOME. The mechanism of action of 12,13-diHOME is similar to exercise (i.e., NOS1) to produce physiological remodeling, as in the “athlete's” heart. This study uncovers a novel mechanism for metabolic induction of physiological remodeling that underlies the endocrine effects of BAT on cardiac function and provides a new mechanism for 12,13-diHOME as a therapeutic modulator for cardiovascular disease.

Here, the study determined that transplantation of BAT (+BAT) improves cardiac function via the release of the lipokine 12,13-diHOME. Sustained overexpression of 12,13-diHOME using tissue nanotransfection negated the deleterious effects of a high-fat diet on cardiac function and remodeling, and acute injection of 12,13-diHOME increased cardiac hemodynamics via direct effects on the cardiomyocyte. Furthermore, incubation of cardiomyocytes with 12,13-diHOME increased mitochondrial respiration. The effects of 12,13-diHOME were absent in NOS1^{-/-} mice and cardiomyocytes. This study also provides the first evidence that 12,13-diHOME is decreased in human patients with heart disease. These results identify an endocrine role for BAT to enhance cardiac function that is mediated by regulation of calcium cycling via 12,13-diHOME and NOS1.

In summary, the inventors have identified, for the first time, a role for brown adipose tissue (BAT) to mediate cardiac function via the release of the lipokine 12,13-diHOME. In addition, increasing 12,13-diHOME increases cardiac function and cardiomyocyte respiration via NOS1. Finally, this is the first study to determine that 12,13-diHOME levels are decreased in patients with heart disease. Thus, this study uncovers a novel mechanism for metabolic induction of physiological remodeling that underlies the endocrine effects of BAT on cardiac function.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

SEQUENCES
(nucleotide sequence of mouse Ephx1)
SEQ ID NO: 1
ATGTGGCTGGAACTCATCCTGGCTTCTGTGCTGGGCTTTGTCATCTACTGGTTTGTCT
CCCGGGACAAGGAGGAGACCTTACCACTTGAAGATGGGTGGTGGGGCCCAGGGTC
AAAGCCATCAGCCAAAGAAGATGAGAGCATCCGGCCCTTCAAGGTGGAAACATCA
GATGAGGAGATCAAGGACTTGCACCAGAGGATAGATAGGTTCCGGGCATCCCCACC
TTTGGAGGGCAGTCGCTTCCACTATGGCTTCAACTCCAGCTACCTGAAGAAAGTGGT
GTCCTTCTGGAGGAATGAGTTTGACTGGAGGAAGCAGGTGGAGATCCTCAACCAAT
ACCCACACTTTAAGACCAAGATTGAAGGGCTGGACATCCACTTCATCCACGTGAAA
CCTCCCCAGCTGCCCTCAGGCCGCACTCCAAAGCCCTTGCTGATGGTGCACGGCTGG
CCTGGCTCCTTCTATGAGTTCTACAAGATTATCCCACTGCTGACAGACCCCAAGACC
CACGGCCTGAGTGATGAGCACGTGTTTGAAGTCATCTGTCCCTCAATTCCTGGCTAT
GGCTTCTCAGAGGCATCCAGCAAGAAAGGTTTAAATTCGGTGGCCACTGCGAGGAT
CTTCTACAAGCTGATGTCACGGCTGGGCTTCCAGAAGTTCTACATTCAAGGCGGCGA
CTGGGGGTCTCTCATCTGCACCAACATAGCCCAGATGGTGCCCAACCACGTGAAAG
GTTTGCACTTGAATATGTCTTTCATTTCAAGAAACATTTATTCCCTGACCCCTCTCCT
GGGCCAACGTTTTGGGAGATTTCTTGGCTACACAGAGAAGGATCTGGAGCTCTTGT
ACCCATTCAAGGAAAAGGTTTTCTACAACATCATGAGGGAGAGTGGCTACTTACAC
ATCCAGGCCACCAAGCCGGACACTGTGGGCTGTGCTCTGAATGACTCTCCTGTGGG
CCTGGCTGCCTACATCTTAGAGAAGTTCTCCACCTGGACCAAGTCAGAATACCGTGA
ACTGGAGGATGGAGGCCTGGAGAGGAAGTTCTCCCTGGAAGATCTGCTGACTAACA
TCATGATCTACTGGACGACAGGAACCATTGTCTCCTCCCAGCGCTTCTACAAGGAAA
ACTTGGGCCAGGGTGTCATGGTCCATAGACATGAGGGGATGAAGGTCTTTGTGCCC
ACTGGCTATTCAGCCTTCCCTTCTGAGATCCTGCATGCCCCAGAAAAGTGGGTGAAG
GTCAAGTACCCCAAACTCATCTCCTATTCCTACATGGAACGTGGGGGCCACTTTGCT
GCCTTCGAAGAGCCCAAGCTTCTGGCCCAGGACATCCGCAAGTTCGTGTCCCTGGCT
GAGCTGCAG
(protein sequence of mouse Ephx1)
SEQ ID NO: 2
MWLELILASVLGFVIYWFVSRDKEETLPLEDGWWGPGSKPSAKEDESIRPFKVETSDEEI
KDLHQRIDRFRASPPLEGSRFHYGFNSSYLKKVVSFWRNEFDWRKQVEILNQYPHFKTK
TEGLDIHFIHVKPPQLPSGRTPKPLLMVHGWPGSFYEFYKIIPLLTDPKTHGLSDEHVFEVI
CPSIPGYGFSEASSKKGLNSVATARIFYKLMSRLGFQKFYIQGGDWGSLICTNIAQMVPN
HVKGLHLNMSFISRNIYSLTPLLGQRFGRFLGYTEKDLELLYPFKEKVFYNIMRESGYLH
IQATKPDTVGCALNDSPVGLAAYILEKFSTWTKSEYRELEDGGLERKFSLEDLLTNIMIY
WTTGTIVSSQRFYKENLGQGVMVHRHEGMKVFVPTGYSAFPSEILHAPEKWVKVKYP
KLISYSYMERGGHFAAFEEPKLLAQDIRKFVSLAELQ
(nucleic acid sequence of mouse Ephx 2)
SEQ ID NO: 3
ATGGCGCTGCGTGTAGCCGCGTTCGACCTTGACGGAGTGCTGGCCCTCCCCTCTATC
GCCGGGGCTTTCCGCCGCAGCGAAGAGGCCCTGGCACTGCCTAGAGACTTCCTGCT
TGGTGCGTACCAGACGGAATTCCCAGAGGGACCCACTGAGCAACTCATGAAAGGGA
AGATCACATTTTCGCAGTGGGTACCACTCATGGATGAAAGCTACAGGAAGTCCTCC
AAAGCCTGTGGAGCCAATCTACCTGAGAATTTCTCCATAAGTCAAATATTCAGCCA
AGCTATGGCAGCAAGAAGCATCAACCGCCCCATGCTTCAGGCAGCCATTGCTCTCA
AAAAGAAAGGATTCACAACATGCATTGTCACCAACAACTGGCTGGACGACGGAGA
CAAGAGAGACAGCCTGGCCCAGATGATGTGTGAGCTGAGCCAACACTTTGACTTCC
TGATAGAGTCCTGTCAGGTTGGGATGATCAAGCCTGAGCCTCAGATCTACAATTTTT
TACTGGATACCCTGAAGGCAAAACCCAATGAGGTTGTTTTCCTAGATGACTTTGGAA
GTAATCTGAAGCCAGCCCGTGACATGGGGATGGTTACCATCCTGGTCCACAACACA
GCCTCCGCTCTGAGAGAACTGGAGAAGGTCACAGGGACACAGTTTCCTGAGGCCCC
ACTGCCAGTCCCATGCAATCCAAATGACGTCAGCCATGGATATGTGACAGTGAAGC
CAGGGATCCGCCTGCATTTTGTGGAGATGGGCTCTGGCCCTGCCCTATGCCTTTGCC
ATGGGTTTCCTGAGAGCTGGTTTTCTTGGAGGTACCAGATCCCTGCTCTGGCCCAGG
CAGGCTTTCGTGTTCTGGCTATAGACATGAAAGGCTATGGAGACTCATCTTCTCCTC
CTGAAATAGAAGAATATGCCATGGAATTGCTGTGTAAGGAGATGGTGACATTCCTG
GATAAGCTGGGAATCCCTCAAGCAGTGTTCATTGGCCATGACTGGGCTGGTGTGAT
GGTGTGGAACATGGCTCTCTTCTACCCTGAGAGAGTGAGGGCTGTGGCCAGTTTGA
ACACGCCGTTTATGCCACCAGATCCTGATGTGTCTCCCATGAAAGTTATCCGATCTA
TCCCAGTTTTCAATTATCAGCTGTACTTTCAAGAACCAGGAGTGGCCGAGGCTGAAC
TGGAGAAGAACATGAGTCGGACTTTCAAAAGCTTCTTCCGAGCCAGTGATGAGACA
GGTTTCATCGCTGTGCATAAAGCCACTGAAATAGGGGGAATCCTTGTGAATACTCC
AGAAGATCCCAACCTCAGCAAAATTACTACTGAGGAAGAAATAGAGTTTTACATAC
AGCAGTTCAAGAAGACTGGCTTCAGAGGTCCTCTGAACTGGTACCGGAACACAGAA
AGAAACTGGAAGTGGAGCTGTAAAGGGTTGGGACGAAAGATCTTGGTCCCAGCCCT
GATGGTCACAGCTGAGAAGGACATTGTACTCCGTCCTGAAATGTCCAAGAACATGG
AAAAGTGGATCCCTTTCCTGAAAAGGGGACACATTGAAGACTGTGGTCACTGGACA
CAGATAGAGAAACCAACTGAGGTGAACCAGATTCTCATCAAGTGGCTGCAGACTGA
AGTCCAGAACCCATCAGTGACCTCCAAGATT
(protein sequence of mouse Ephx 2)
SEQ ID NO: 4
MALRVAAFDLDGVLALPSIAGAFRRSEEALALPRDFLLGAYQTEFPEGPTEQLMKGKIT
FSQWVPLMDESYRKSSKACGANLPENFSISQIFSQAMAARSINRPMLQAAIALKKKGFT
TCIVTNNWLDDGDKRDSLAQMMCELSQHFDFLIESCQVGMIKPEPQIYNFLLDTLKAKP
NEVVFLDDFGSNLKPARDMGMVTILVENTASALRELEKVTGTQFPEAPLPVPCNPNDV
SHGYVTVKPGIRLHFVEMGSGPALCLCHGFPESWFSWRYQIPALAQAGFRVLAIDMKG
YGDSSSPPEJEEYAMELLCKEMVTFLDKLGIPQAVFIGHDWAGVMVWNMALFYPERVR
AVASLNTPFMPPDPDVSPMKVIRSIPVFNYQLYFQEPGVAEAELEKNMSRTFKSFFRASD
ETGFIAVHKATEIGGILVNTPEDPNLSKITTEEEIEFYIQQFKKTGFRGPLNWYRNTERNW
KWSCKGLGRKILVPALMVTAEKDIVLRPEMSKNMEKWIPFLKRGHIEDCGHWTQIEKP
TEVNOILIKWLQTEVQNPSVTSKI
(nucleic acid sequence of mouse Ephx 3)
SEQ ID NO: 5
ATGAGAGGTGGCAGTATCTGTCCGAGCCGCGCATCTGTTTCCTCGACCGGTCCAGTG
GACTCCGATAGCACGGTGGAATCCCAAAACAAAGGTGGTGGGCTGCTGGCGCCAGC
ACCCCTGGCTCAGTCTCCCGACCACGGCGGCTCTGTAGTCGTCCCAGAGCGCAAGG
ATATGCCGGAGTTTGTGGTGACAGCGCTACTCGCACCCTCACGCCTCTCGCTGAAGC
TGCTGCGAGCGCTGGTGATGAGCCTGGTGTACTTGGCTGCCTTGGTGGCCGCGTTTG
TCTACAGCTGCATCGCGCTCACCCATGTGATGTGCCGTCCTCGCAGGGGCTGCTGCG
GCCGCCAGAGGTTGTCTCCCCCAGAGTGCCTGAGAGACCCCACGCTGGGCGAGCAT
TGCTTTCTAACCCTCAGGGTGAGTGTTCCTCCCGTGAAGAGTTCCGGCCTGCGTCTG
CACTATGTCTCTGCTGGTCATGGCAATGGGCCACTCATGCTATTTCTGCATGGCTTC
CCAGAGAACTGGTTCAGCTGGCGCTACCAGCTGCGAGAGTTTCAGAGCCATTTCCA
TGTCGTGGCTGTAGACATGCGTGGTTATAGTCCCTCTGATGCTCCAAAGGAAGTGGA
TTGTTACACCATTGACTTGTTGTTGGATGACATCAAGGATACCATCCTAGGCCTGGG
GTACTCCAAGTGCATCCTCGTGAGCCACGACTGGGGGGCCTCCCTTGCCTGGGAGTT
CTCCATCTACTACCCATCCCTAGTGGAGCGTATGGTTGTGGCCAATGGTCCTCCCAT
GTCAGTGATCCAAGAATACTCAATCCACCACATCGGCCAGATATTCCGATCAAACT
ACATGTTCCTGTTCCAGCTTCCCTGGCTGCCAGAGAAGCTGTTGTCTATGTCTGACTT
CCAGATTCTCAAAGACACATTCACTCACCGCAAGAACGGCATCCCAGGACTGACTC
CTTCTGAACTTGAAGCATTCCTTTATCACTTCTCACAACCTGGATGCCTCACTGGGC
CCATCAACTACTACAGGAACGTGTTCAGGAACTTCCCCCTGGAGCCCAAGAAACTG
TCAACACCCACGCTGTTGCTGTGGGGGGAAAAAGACTTCGCCTTCCAGCAGGGGCT
GGTGGAAGCCATTGGAAGACACTTTGTGCCCGGCCGGTTGGAAAGCCACATTTTGC
CAGGCAGTGGGCACTGGATTCCACAGAGCCATCCTCAGGAGATGCATCAGTACATG
TGGGCCTTCTTGCAAGACCTGCTGGGC
(protein sequence of mouse Ephx 3)
SEQ ID NO: 6
MRGGSICPSRASVSSTGPVDSDSTVESQNKGGGLLAPAPLAQSPDHGGSVVVPERKDMP
EFVVTALLAPSRLSLKLLRALVMSLVYLAALVAAFVYSCIALTHVMCRPRRGCCGRQR
LSPPECLRDPTLGEHCFLTLRVSVPPVKSSGLRLHYVSAGHGNGPLMLFLHGFPENWFS
WRYQLREFQSHFHVVAVDMRGYSPSDAPKEVDCYTIDLLLDDIKDTILGLGYSKCILVS
HDWGASLAWEFSIYYPSLVERMVVANGPPMSVIQEYSIHHIGQIFRSNYMFLFQLPWLP
EKLLSMSDFQILKDTFTHRKNGIPGLTPSELEAFLYHFSQPGCLTGPINYYRNVFRNFPLE
PKKLSTPTLLLWGEKDFAFQQGLVEAIGRHFVPGRLESHILPGSGHWIPQSHPQEMHQY
MWAFLQDLLG
(nucleic acid sequence of mouse Ephx 4)
SEQ ID NO: 7
ATGGCCCCGCCGCGCCCGCCCCGCCTGCTGCCCGCGCTGCGCGCCCTGCTCTACTGG
TCCCTGGTGTACGGCTACTGCGGGCTGTGCGCCTCCGTCCACCTGCTCAAACTTTTG
TGGAGCATCGGTAGGGCGCCGGCGCAGACCTTCCGCCGGGCGGCCCGGGCCAACCC
TCCGGCGTGCCTGAACGACCCCTCCTTAGGGACTCACTGCTACGTGCGCATCAAGG
ACTCCGGGTTAAGATTTCACTATGTTGCTGCTGGAGAAAGGGGCAAACCGCTCATG
CTGCTGCTTCATGGATTTCCAGAATTCTGGTATTCTTGGCGCCATCAACTGAGAGAA
TTTAAAAGCGAATACAGGGTTGTTGCATTGGATTTGAGAGGTTATGGAGAGTCTGA
TGCACCTGCTCATCAAGAGAGTTACAAACTGGACTGTCTAATTGCAGACATAAAGG
ATATTTTGGACTCCTTAGGGTATAGCAAATGTGTCCTGATCGGCCATGACTGGGGAG
GCATGATTGCCTGGCTGATTGCTGTCTGCTACCCTGAGATGATAATGAAGCTCATTG
TTATTAACTTCCCACATCCAAGTGTATTTACAGAGTATATACTGCGGCATCCTGCCC
GTTCTCAATTAATGATTTTAAGGCTTTGAAACATCTGTTTACCAGTCAGAGTACTGG
CATTGGAAGGAAAGGACGCCAGCTGACAACAGAAGATCTAGAAGCTTACGTTTATG
TCTTTTCTCAGCCTGGAGCACTAAGTGGTCCAATTAACCATTATCGAAACATTTTCA
GCTGCCTGCCTCTCAAACATCACATGGTGACCACCCCAACACTGCTTCTGTGGGGAG
AGGAAGATGCGTTTATGGAGGTAGAGATGGCCGAGGTCACAAAGATTTATGTTAAA
AACTATTTCAGACTCACCATTTTGTCAGAAGGTAGCCACTGGCTTCAGCAAGACCAG
CCTGACATAGTGAATGGACTGATATGGGCATTCCTGAAGGAAGAAACAAGGAGAG
AC
(protein sequence of mouse Ephx 4)
SEQ ID NO: 8
MAPPRPPRLLPALRALLYWSLVYGYCGLCASVHLLKLLWSIGRAPAQTFRRAARANPP
ACLNDPSLGTHCYVRIKDSGLRFHYVAAGERGKPLMLLLHGFPEFWYSWRHQLREFKS
EYRVVALDLRGYGESDAPAHQESYKLDCLIADIKDILDSLGYSKCVLIGHDWGGMIAW
LIAVCYPEMIMKLIVINFPHPSVFTEYILRHPAQLFRSSFYYFFQIPRFPEFMFSINDFKALK
HILFTSQSTGIGRKGRQLTTEDLEAYVYVFSQPGALSGPINHYRNIFSCLPLKHHMVTTPT
LLLWGEEDAFMEVEMAEVTKIYVKNYFRLTILSEGSHWLQQDQPDIVNGLIWAFLKEE
TRRD
(protein sequence of human Ephx 1)
SEQ ID NO: 9
MWLEILLTSVLGFAIYWFISRDKEETLPLEDGWWGPGTRSAAREDDSIRPFKVETSDEEI
HDLHQRIDKFRFTPPLEDSCFHYGFNSNYLKKVISYWRNEFDWKKQVEILNRYPHFKTK
IEGLDIHFIHVKPPQLPAGHTPKPLLMVHGWPGSFYEFYKIIPLLTDPKNHGLSDEHVFEV
ICPSIPGYGFSEASSKKGFNSVATARIFYKLMLRLGFQEFYIQGGDWGSLICTNMAQLVP
SHVKGLHLNMALVLSNFSTLTLLLGQRFGRFLGLTERDVELLYPVKEKVFYSLMRESG
YMHIQCTKPDTVGSALNDSPVGLAAYILEKFSTWTNTEFRYLEDGGLERKFSLDDLLIN
VMLYWTTGTIISSQRFYKENLGQGWMTQKHERMKVYVPTGFSAFPFELLHTPEKWVRF
KYPKLISYSYMVRGGHFAAFEEPELLAQDIRKFLSVLERQ
(protein sequence of human Ephx 2)
SEQ ID NO: 10
MTLRAAVFDLDGVLALPAVFGVLGRTEEALALPRGLLNDAFQKGGPEGATTRLMKGEI
TLSQWIPLMEENCRKCSETAKVCLPKNFSIKEIFDKAISARKINRPMLQAALMLRKKGFT
TAILTNTWLDDRAERDGLAQLMCELKMHFDFLIESCQVGMVKPEPQIYKFLLDTLKASP
SEVVELDDIGANLKPARDLGMVTILVQDTDTALKELEKVTGIQLLNTPAPLPTSCNPSD
MSHGYVTVKPRVRLHFVELGSGPAVCLCHGFPESWYSWRYQIPALAQAGYRVLAMD
MKGYGESSAPPEIEEYCMEVLCKEMVTFLDKLGLSQAVFIGHDWGGMLVWYMALFYP
ERVRAVASLNTPFIPANPNMSPLESIKANPVFDYQLYFQEPGVAEAELEQNLSRTFKSLF
RASDESVLSMHKVCEAGGLFVNSPEEPSLSRMVTEEEIQFYVQQFKKSGFRGPLNWYR
NMERNWKWACKSLGRKILIPALMVTAEKDFVLVPQMSQHMEDWIPHLKRGHIEDCGH
WTQMDKPTEVNQILIKWLDSDARNPPVVSKM
(protein sequence of human Ephx 3)
SEQ ID NO: 11
MPELVVTALLAPSRLSLKLLRAFMWSLVFSVALVAAAVYGCIALTHVLCRPRRGCCGR
RRSASPACLSDPSLGEHGFLNLKSSGLRLHYVSAGRGNGPLMLFLHGFPENWFSWRYQ
LREFQSRFHVVAVDLRGYGPSDAPRDVDCYTIDLLLVDIKDVILGLGYSKCILVAHDWG
ALLAWHFSIYYPSLVERMVVVSGAPMSVYQDYSLHHISQFFRSHYMFLFQLPWLPEKLL
SMSDFQILKTTLTHRKTGIPCLIPSELEAFLYNFSQPGGLTGPLNYYRNLFRNEPLEPQEL
TTPTLLLWGEKDTYLELGLVEAIGSRFVPGRLEAHILPGIGHWIPQSNPQEMHQYMWAF
LQDLLD
(protein sequence of human Ephx 4)
SEQ ID NO: 12
MARLRDCLPRLMLTLRSLLFWSLVYCYCGLCASIHLLKLLWSLGKGPAQTFRRPAREH
PPACLSDPSLGTHCYVRIKDSGLRFHYVAAGERGKPLMLLLHGFPEFWYSWRYQLREF
KSEYRVVALDLRGYGETDAPIHRQNYKLDCLITDIKDILDSLGYSKCVLIGHDWGGMIA
WLIAICYPEMVMKLIVINFPHPNVFTEYILRHPAQLLKSSYYYFFQIPWFPEFMFSINDFK
VLKHILFTSHSTGIGRKGCQLTTEDLEAYIYVFSQPGALSGPINHYRNIFSCLPLKHHMVT
TPTLLLWGENDAFMEVEMAEVTKIYVKNYFRLTILSEASHWLQQDQPDIVNKLIWTFL
KEETRKKD
SEQ ID NO: 13
CAATGGTTCCTGTCGTCCAGTAG
SEQ ID NO: 14
AGTTCTCCACCTGGACCAAGTC
SEQ ID NO: 15
ATCTGGTGGCATAAACGGCGTG
SEQ ID NO: 16
CCCTCAAGCAGTGTTCATTGGC
SEQ ID NO: 17
GGAAATGGCTCTGAAACTCTCGC
SEQ ID NO: 18
GCACTATGTCTCTGCTGGTCATG
SEQ ID NO: 19
GCCATCTCTACCTCCATAAACGC
SEQ ID NO: 20
TCTCAGCCTGGAGCACTAAGTG
SEQ ID NO: 21
ACCACTCATGGATGAAAGCTACA
SEQ ID NO: 22
TCAGGTAGATTGGCTCCACAG
SEQ ID NO: 23
GTTTTCCACCTGGACCAATACGG
SEQ ID NO: 24
TGGTGCCTGTTGTCCAGTAGAG
SEQ ID NO: 25
AGCCTCTTCAGAGCAAGCGATG
SEQ ID NO: 26
GGATTTCCTCCTCAGTGACCATC
SEQ ID NO: 27
TGTTGTGGCTGTGGACTTGCGA
SEQ ID NO: 28
GCCACAAGGATGCACTTCGAGT
SEQ ID NO: 29
CTGCTGGAGAAAGAGGCAAACC
SEQ ID NO: 30
CCATAACCTCTCAAATCCAGTGC

1. A method for increasing a level of a lipokine in a subject, comprising administering to the subject an effective amount of an epoxide hydrolase (Ephx) polypeptide or a polynucleotide encoding the Ephx polypeptide.

2. The method of claim 1, wherein the Ephx polypeptide comprises an Ephx1 polypeptide or an Ephx2 polypeptide.

3. The method of claim 1, wherein the Ephx polypeptide comprises an Ephx1 polypeptide and an Ephx2 polypeptide.

4. The method of claim 1, wherein the polynucleotide encoding the Ephx polypeptide comprises a nucleic acid sequence at least about 80% identical to SEQ ID NO:1, SEQ ID NO:3, SEQ ID NO:5, or SEQ ID NO:7.

5. The method of claim 1, wherein the polynucleotide is contained in a vector.

6. The method of claim 5, wherein the vector is a viral vector.

7. The method of claim 6, wherein the viral vector is an adeno-associated virus (AAV) vector.

8. The method of claim 1, wherein the polynucleotide is an mRNA.

9. The method of claim 8, wherein the mRNA is contained in a nanoparticle.

10. The method of claim 1, wherein the lipokine comprises 12,13-diHOME.

11. A method of treating a cardiovascular disease, comprising administering to a subject in need thereof a therapeutically effective amount of an epoxide hydrolase (Ephx) polypeptide or a polynucleotide that encodes the Ephx polypeptide.

12. The method of claim 11, wherein the Ephx polypeptide comprises an Ephx1 polypeptide or an Ephx2 polypeptide.

13. The method of claim 11, wherein the Ephx polypeptide comprises an Ephx1 polypeptide and an Ephx2 polypeptide.

14. The method of claim 11, wherein the polynucleotide comprises a nucleic acid sequence at least about 80% identical to SEQ ID NO: 1, 3, 5, or 7.

15.-19. (canceled)

20. The method of claim 11, wherein a level of a lipokine is increased in comparison to a reference control.

21. The method of claim 11, wherein the lipokine comprises 12,13-diHOME.

22. A method of treating an inflammatory disease, comprising administering to a subject in need thereof a therapeutically effective amount of an epoxide hydrolase (Ephx) polypeptide or a polynucleotide that encodes the Ephx polypeptide.

23.-30. (canceled)

31. The method of claim 22, wherein a level of a lipokine is increased in comparison to a reference control.

32. The method of claim 31, wherein the lipokine comprises 12,13-diHOME.

33. The method of claim 22, wherein the inflammatory disease is type 2 diabetes or nonalcoholic fatty liver disease.


SEQUENCE LISTING (30 sequences)

SEQ ID NO: 1 - DNA, 1365 bp, Mus musculus
atgtggctggaactcatcctggcttctgtgctgggctttgtcatctactggtttgtctcccgggacaaggaggagaccttaccacttgaagatgggtggtggggcccagggtcaaagccatcagccaaagaagatgagagcatccggcccttcaaggtggaaacatcagatgaggagatcaaggacttgcaccagaggatagataggttccgggcatccccacctttggagggcagtcgcttccactatggcttcaactccagctacctgaagaaagtggtgtccttctggaggaatgagtttgactggaggaagcaggtggagatcctcaaccaatacccacactttaagaccaagattgaagggctggacatccacttcatccacgtgaaacctccccagctgccctcaggccgcactccaaagcccttgctgatggtgcacggctggcctggctccttctatgagttctacaagattatcccactgctgacagaccccaagacccacggcctgagtgatgagcacgtgtttgaagtcatctgtccctcaattcctggctatggcttctcagaggcatccagcaagaaaggtttaaattcggtggccactgcgaggatcttctacaagctgatgtcacggctgggcttccagaagttctacattcaaggcggcgactgggggtctctcatctgcaccaacatagcccagatggtgcccaaccacgtgaaaggtttgcacttgaatatgtctttcatttcaagaaacatttattccctgacccctctcctgggccaacgttttgggagatttcttggctacacagagaaggatctggagctcttgtacccattcaaggaaaaggttttctacaacatcatgagggagagtggctacttacacatccaggccaccaagccggacactgtgggctgtgctctgaatgactctcctgtgggcctggctgcctacatcttagagaagttctccacctggaccaagtcagaataccgtgaactggaggatggaggcctggagaggaagttctccctggaagatctgctgactaacatcatgatctactggacgacaggaaccattgtctcctcccagcgcttctacaaggaaaacttgggccagggtgtcatggtccatagacatgaggggatgaaggtctttgtgcccactggctattcagccttcccttctgagatcctgcatgccccagaaaagtgggtgaaggtcaagtaccccaaactcatctcctattcctacatggaacgtgggggccactttgctgccttcgaagagcccaagcttctggcccaggacatccgcaagttcgtgtccctggctgagctgcag

SEQ ID NO: 2 - protein, 455 aa, Mus musculus
MWLELILASVLGFVIYWFVSRDKEETLPLEDGWWGPGSKPSAKEDESIRPFKVETSDEEIKDLHQRIDRFRASPPLEGSRFHYGFNSSYLKKVVSFWRNEFDWRKQVEILNQYPHFKTKIEGLDIHFIHVKPPQLPSGRTPKPLLMVHGWPGSFYEFYKIIPLLTDPKTHGLSDEHVFEVICPSIPGYGFSEASSKKGLNSVATARIFYKLMSRLGFQKFYIQGGDWGSLICTNIAQMVPNHVKGLHLNMSFISRNIYSLTPLLGQRFGRFLGYTEKDLELLYPFKEKVFYNIMRESGYLHIQATKPDTVGCALNDSPVGLAAYILEKFSTWTKSEYRELEDGGLERKFSLEDLLTNIMIYWTTGTIVSSQRFYKENLGQGVMVHRHEGMKVFVPTGYSAFPSEILHAPEKWVKVKYPKLISYSYMERGGHFAAFEEPKLLAQDIRKFVSLAELQ

SEQ ID NO: 3 - DNA, 1662 bp, Mus musculus
atggcgctgcgtgtagccgcgttcgaccttgacggagtgctggccctcccctctatcgccggggctttccgccgcagcgaagaggccctggcactgcctagagacttcctgcttggtgcgtaccagacggaattcccagagggacccactgagcaactcatgaaagggaagatcacattttcgcagtgggtaccactcatggatgaaagctacaggaagtcctccaaagcctgtggagccaatctacctgagaatttctccataagtcaaatattcagccaagctatggcagcaagaagcatcaaccgccccatgcttcaggcagccattgctctcaaaaagaaaggattcacaacatgcattgtcaccaacaactggctggacgacggagacaagagagacagcctggcccagatgatgtgtgagctgagccaacactttgacttcctgatagagtcctgtcaggttgggatgatcaagcctgagcctcagatctacaattttttactggataccctgaaggcaaaacccaatgaggttgttttcctagatgactttggaagtaatctgaagccagcccgtgacatggggatggttaccatcctggtccacaacacagcctccgctctgagagaactggagaaggtcacagggacacagtttcctgaggccccactgccagtcccatgcaatccaaatgacgtcagccatggatatgtgacagtgaagccagggatccgcctgcattttgtggagatgggctctggccctgccctatgcctttgccatgggtttcctgagagctggttttcttggaggtaccagatccctgctctggcccaggcaggctttcgtgttctggctatagacatgaaaggctatggagactcatcttctcctcctgaaatagaagaatatgccatggaattgctgtgtaaggagatggtgacattcctggataagctgggaatccctcaagcagtgttcattggccatgactgggctggtgtgatggtgtggaacatggctctcttctaccctgagagagtgagggctgtggccagtttgaacacgccgtttatgccaccagatcctgatgtgtctcccatgaaagttatccgatctatcccagttttcaattatcagctgtactttcaagaaccaggagtggccgaggctgaactggagaagaacatgagtcggactttcaaaagcttcttccgagccagtgatgagacaggtttcatcgctgtgcataaagccactgaaatagggggaatccttgtgaatactccagaagatcccaacctcagcaaaattactactgaggaagaaatagagttttacatacagcagttcaagaagactggcttcagaggtcctctgaactggtaccggaacacagaaagaaactggaagtggagctgtaaagggttgggacgaaagatcttggtcccagccctgatggtcacagctgagaaggacattgtactccgtcctgaaatgtccaagaacatggaaaagtggatccctttcctgaaaaggggacacattgaagactgtggtcactggacacagatagagaaaccaactgaggtgaaccagattctcatcaagtggctgcagactgaagtccagaacccatcagtgacctccaagatt

SEQ ID NO: 4 - protein, 554 aa, Mus musculus
MALRVAAFDLDGVLALPSIAGAFRRSEEALALPRDFLLGAYQTEFPEGPTEQLMKGKITFSQWVPLMDESYRKSSKACGANLPENFSISQIFSQAMAARSINRPMLQAAIALKKKGFTTCIVTNNWLDDGDKRDSLAQMMCELSQHFDFLIESCQVGMIKPEPQIYNFLLDTLKAKPNEVVFLDDFGSNLKPARDMGMVTILVHNTASALRELEKVTGTQFPEAPLPVPCNPNDVSHGYVTVKPGIRLHFVEMGSGPALCLCHGFPESWFSWRYQIPALAQAGFRVLAIDMKGYGDSSSPPEIEEYAMELLCKEMVTFLDKLGIPQAVFIGHDWAGVMVWNMALFYPERVRAVASLNTPFMPPDPDVSPMKVIRSIPVFNYQLYFQEPGVAEAELEKNMSRTFKSFFRASDETGFIAVHKATEIGGILVNTPEDPNLSKITTEEEIEFYIQQFKKTGFRGPLNWYRNTERNWKWSCKGLGRKILVPALMVTAEKDIVLRPEMSKNMEKWIPFLKRGHIEDCGHWTQIEKPTEVNQILIKWLQTEVQNPSVTSKI

SEQ ID NO: 5 - DNA, 1272 bp, Mus musculus
atgagaggtggcagtatctgtccgagccgcgcatctgtttcctcgaccggtccagtggactccgatagcacggtggaatcccaaaacaaaggtggtgggctgctggcgccagcacccctggctcagtctcccgaccacggcggctctgtagtcgtcccagagcgcaaggatatgccggagtttgtggtgacagcgctactcgcaccctcacgcctctcgctgaagctgctgcgagcgctggtgatgagcctggtgtacttggctgccttggtggccgcgtttgtctacagctgcatcgcgctcacccatgtgatgtgccgtcctcgcaggggctgctgcggccgccagaggttgtctcccccagagtgcctgagagaccccacgctgggcgagcattgctttctaaccctcagggtgagtgttcctcccgtgaagagttccggcctgcgtctgcactatgtctctgctggtcatggcaatgggccactcatgctatttctgcatggcttcccagagaactggttcagctggcgctaccagctgcgagagtttcagagccatttccatgtcgtggctgtagacatgcgtggttatagtccctctgatgctccaaaggaagtggattgttacaccattgacttgttgttggatgacatcaaggataccatcctaggcctggggtactccaagtgcatcctcgtgagccacgactggggggcctcccttgcctgggagttctccatctactacccatccctagtggagcgtatggttgtggccaatggtcctcccatgtcagtgatccaagaatactcaatccaccacatcggccagatattccgatcaaactacatgttcctgttccagcttccctggctgccagagaagctgttgtctatgtctgacttccagattctcaaagacacattcactcaccgcaagaacggcatcccaggactgactccttctgaacttgaagcattcctttatcacttctcacaacctggatgcctcactgggcccatcaactactacaggaacgtgttcaggaacttccccctggagcccaagaaactgtcaacacccacgctgttgctgtggggggaaaaagacttcgccttccagcaggggctggtggaagccattggaagacactttgtgcccggccggttggaaagccacattttgccaggcagtgggcactggattccacagagccatcctcaggagatgcatcagtacatgtgggccttcttgcaagacctgctgggc

SEQ ID NO: 6 - protein, 424 aa, Mus musculus
MRGGSICPSRASVSSTGPVDSDSTVESQNKGGGLLAPAPLAQSPDHGGSVVVPERKDMPEFVVTALLAPSRLSLKLLRALVMSLVYLAALVAAFVYSCIALTHVMCRPRRGCCGRQRLSPPECLRDPTLGEHCFLTLRVSVPPVKSSGLRLHYVSAGHGNGPLMLFLHGFPENWFSWRYQLREFQSHFHVVAVDMRGYSPSDAPKEVDCYTIDLLLDDIKDTILGLGYSKCILVSHDWGASLAWEFSIYYPSLVERMVVANGPPMSVIQEYSIHHIGQIFRSNYMFLFQLPWLPEKLLSMSDFQILKDTFTHRKNGIPGLTPSELEAFLYHFSQPGCLTGPINYYRNVFRNFPLEPKKLSTPTLLLWGEKDFAFQQGLVEAIGRHFVPGRLESHILPGSGHWIPQSHPQEMHQYMWAFLQDLLG

SEQ ID NO: 7 - DNA, 1077 bp, Mus musculus
atggccccgccgcgcccgccccgcctgctgcccgcgctgcgcgccctgctctactggtccctggtgtacggctactgcgggctgtgcgcctccgtccacctgctcaaacttttgtggagcatcggtagggcgccggcgcagaccttccgccgggcggcccgggccaaccctccggcgtgcctgaacgacccctccttagggactcactgctacgtgcgcatcaaggactccgggttaagatttcactatgttgctgctggagaaaggggcaaaccgctcatgctgctgcttcatggatttccagaattctggtattcttggcgccatcaactgagagaatttaaaagcgaatacagggttgttgcattggatttgagaggttatggagagtctgatgcacctgctcatcaagagagttacaaactggactgtctaattgcagacataaaggatattttggactccttagggtatagcaaatgtgtcctgatcggccatgactggggaggcatgattgcctggctgattgctgtctgctaccctgagatgataatgaagctcattgttattaacttcccacatccaagtgtatttacagagtatatactgcggcatcctgcccagctgttcagatccagcttttattacttcttccaaataccacgcttcccagaatttatgttctcaattaatgattttaaggctttgaaacatctgtttaccagtcagagtactggcattggaaggaaaggacgccagctgacaacagaagatctagaagcttacgtttatgtcttttctcagcctggagcactaagtggtccaattaaccattatcgaaacattttcagctgcctgcctctcaaacatcacatggtgaccaccccaacactgcttctgtggggagaggaagatgcgtttatggaggtagagatggccgaggtcacaaagatttatgttaaaaactatttcagactcaccattttgtcagaaggtagccactggcttcagcaagaccagcctgacatagtgaatggactgatatgggcattcctgaaggaagaaacaaggagagac

SEQ ID NO: 8 - protein, 359 aa, Mus musculus
MAPPRPPRLLPALRALLYWSLVYGYCGLCASVHLLKLLWSIGRAPAQTFRRAARANPPACLNDPSLGTHCYVRIKDSGLRFHYVAAGERGKPLMLLLHGFPEFWYSWRHQLREFKSEYRVVALDLRGYGESDAPAHQESYKLDCLIADIKDILDSLGYSKCVLIGHDWGGMIAWLIAVCYPEMIMKLIVINFPHPSVFTEYILRHPAQLFRSSFYYFFQIPRFPEFMFSINDFKALKHLFTSQSTGIGRKGRQLTTEDLEAYVYVFSQPGALSGPINHYRNIFSCLPLKHHMVTTPTLLLWGEEDAFMEVEMAEVTKIYVKNYFRLTILSEGSHWLQQDQPDIVNGLIWAFLKEETRRD

SEQ ID NO: 9 - protein, 455 aa, Homo sapiens
MWLEILLTSVLGFAIYWFISRDKEETLPLEDGWWGPGTRSAAREDDSIRPFKVETSDEEIHDLHQRIDKFRFTPPLEDSCFHYGFNSNYLKKVISYWRNEFDWKKQVEILNRYPHFKTKIEGLDIHFIHVKPPQLPAGHTPKPLLMVHGWPGSFYEFYKIIPLLTDPKNHGLSDEHVFEVICPSIPGYGFSEASSKKGFNSVATARIFYKLMLRLGFQEFYIQGGDWGSLICTNMAQLVPSHVKGLHLNMALVLSNFSTLTLLLGQRFGRFLGLTERDVELLYPVKEKVFYSLMRESGYMHIQCTKPDTVGSALNDSPVGLAAYILEKFSTWTNTEFRYLEDGGLERKFSLDDLLTNVMLYWTTGTIISSQRFYKENLGQGWMTQKHERMKVYVPTGFSAFPFELLHTPEKWVRFKYPKLISYSYMVRGGHFAAFEEPELLAQDIRKFLSVLERQ

SEQ ID NO: 10 - protein, 555 aa, Homo sapiens
MTLRAAVFDLDGVLALPAVFGVLGRTEEALALPRGLLNDAFQKGGPEGATTRLMKGEITLSQWIPLMEENCRKCSETAKVCLPKNFSIKEIFDKAISARKINRPMLQAALMLRKKGFTTAILTNTWLDDRAERDGLAQLMCELKMHFDFLIESCQVGMVKPEPQIYKFLLDTLKASPSEVVFLDDIGANLKPARDLGMVTILVQDTDTALKELEKVTGIQLLNTPAPLPTSCNPSDMSHGYVTVKPRVRLHFVELGSGPAVCLCHGFPESWYSWRYQIPALAQAGYRVLAMDMKGYGESSAPPEIEEYCMEVLCKEMVTFLDKLGLSQAVFIGHDWGGMLVWYMALFYPERVRAVASLNTPFIPANPNMSPLESIKANPVFDYQLYFQEPGVAEAELEQNLSRTFKSLFRASDESVLSMHKVCEAGGLFVNSPEEPSLSRMVTEEEIQFYVQQFKKSGFRGPLNWYRNMERNWKWACKSLGRKILIPALMVTAEKDFVLVPQMSQHMEDWIPHLKRGHIEDCGHWTQMDKPTEVNQILIKWLDSDARNPPVVSKM

SEQ ID NO: 11 - protein, 360 aa, Homo sapiens
MPELVVTALLAPSRLSLKLLRAFMWSLVFSVALVAAAVYGCIALTHVLCRPRRGCCGRRRSASPACLSDPSLGEHGFLNLKSSGLRLHYVSAGRGNGPLMLFLHGFPENWFSWRYQLREFQSRFHVVAVDLRGYGPSDAPRDVDCYTIDLLLVDIKDVILGLGYSKCILVAHDWGALLAWHFSIYYPSLVERMVVVSGAPMSVYQDYSLHHISQFFRSHYMFLFQLPWLPEKLLSMSDFQILKTTLTHRKTGIPCLTPSELEAFLYNFSQPGGLTGPLNYYRNLFRNFPLEPQELTTPTLLLWGEKDTYLELGLVEAIGSRFVPGRLEAHILPGIGHWIPQSNPQEMHQYMWAFLQDLLD

SEQ ID NO: 12 - protein, 362 aa, Homo sapiens
MARLRDCLPRLMLTLRSLLFWSLVYCYCGLCASIHLLKLLWSLGKGPAQTFRRPAREHPPACLSDPSLGTHCYVRIKDSGLRFHYVAAGERGKPLMLLLHGFPEFWYSWRYQLREFKSEYRVVALDLRGYGETDAPIHRQNYKLDCLITDIKDILDSLGYSKCVLIGHDWGGMIAWLIAICYPEMVMKLIVINFPHPNVFTEYILRHPAQLLKSSYYYFFQIPWFPEFMFSINDFKVLKHLFTSHSTGIGRKGCQLTTEDLEAYIYVFSQPGALSGPINHYRNIFSCLPLKHHMVTTPTLLLWGENDAFMEVEMAEVTKIYVKNYFRLTILSEASHWLQQDQPDIVNKLIWTFLKEETRKKD

SEQ ID NO: 13 - DNA, 23 bp, Mus musculus
caatggttcctgtcgtccagtag

SEQ ID NO: 14 - DNA, 22 bp, Mus musculus
agttctccacctggaccaagtc

SEQ ID NO: 15 - DNA, 22 bp, Mus musculus
atctggtggcataaacggcgtg

SEQ ID NO: 16 - DNA, 22 bp, Mus musculus
ccctcaagcagtgttcattggc

SEQ ID NO: 17 - DNA, 23 bp, Mus musculus
ggaaatggctctgaaactctcgc

SEQ ID NO: 18 - DNA, 23 bp, Mus musculus
gcactatgtctctgctggtcatg

SEQ ID NO: 19 - DNA, 23 bp, Mus musculus
gccatctctacctccataaacgc

SEQ ID NO: 20 - DNA, 22 bp, Mus musculus
tctcagcctggagcactaagtg

SEQ ID NO: 21 - DNA, 23 bp, Mus musculus
accactcatggatgaaagctaca

SEQ ID NO: 22 - DNA, 21 bp, Mus musculus
tcaggtagattggctccacag

SEQ ID NO: 23 - DNA, 23 bp, Homo sapiens
gttttccacctggaccaatacgg

SEQ ID NO: 24 - DNA, 22 bp, Homo sapiens
tggtgcctgttgtccagtagag

SEQ ID NO: 25 - DNA, 22 bp, Homo sapiens
agcctcttcagagcaagcgatg

SEQ ID NO: 26 - DNA, 23 bp, Homo sapiens
ggatttcctcctcagtgaccatc

SEQ ID NO: 27 - DNA, 22 bp, Homo sapiens
tgttgtggctgtggacttgcga

SEQ ID NO: 28 - DNA, 22 bp, Homo sapiens
gccacaaggatgcacttcgagt

SEQ ID NO: 29 - DNA, 22 bp, Homo sapiens
ctgctggagaaagaggcaaacc

SEQ ID NO: 30 - DNA, 23 bp, Homo sapiens
ccataacctctcaaatccagtgc